(19) United States
(12) Patent Application Publication
(10) Pub. No.: US 2018/0312844 A1
(43) Pub. Date: Nov. 1, 2018

(54) TREATMENT FOR CANCER METASTASIS

(71) Applicant: The Board of Regents of the UIniversity of Texas System, Austin, TX (US)

(72) Inventors: Sean MORRISON, Dallas, TX (US); Elena PISKOUNOVA, Dallas, TX (US)

(73) Assignee: The Board of Regents of the UIniversity of Texas System, Austin, TX (US)

(21) Appl. No.: 15/566,048

(22) PCT Filed: Apr. 12, 2016
(86) PCT No.: PCT/US16/27039

§ 371 Date: Oct. 12, 2017

Related U.S. Application Data
(60) Provisional application No. 62/146,792, filed on Apr. 13, 2015.

(51) Int. Cl. C12N 15/113; A61K 31/7088; A61K 45/06; A61P 35/04

(52) U.S. Cl. CPC C12N 15/1137; A61K 31/7088; C12N 2320/31; A61P 35/04; C12N 2310/14; A61K 45/06

(57) ABSTRACT

The present disclosure relates to combination therapies for melanoma using anti-folates and/or inhibitors of ALDH1L1, ALDH1L2, or MTHFD1, and in particular, metastatic melanoma. Drugs for use in such combination include MEK inhibitors, BRAF inhibitors and cardiac glycosides.

This application claims benefit of priority to U.S. Provisional Application Ser. No. 62/146,792, filed Apr. 13, 2015, the entire contents of which are hereby incorporated by reference.

### BACKGROUND

### I. Technical Field

The present disclosure relates generally to the fields of medicine and oncology genetics. More particularly, it relates to the combined use of folate inhibitors to treat metastatic melanoma and breast cancer, including methotrexate and inhibitors of ALDH1L1, ALDH1L2, and MTHFD1.

### II. Related Art

Melanoma is a malignant tumor of melanocytes. Melanocytes are cells that produce the dark pigment, melanin, which is responsible for the color of skin. They predominantly occur in skin, but are also found in other parts of the body, including the bowel, oral cavity and the eye. Melanin also protects the deeper layers of the skin from the sun's harmful ultraviolet (UV) rays. When people spend time in the sunlight, the melanocytes make more melanin and cause the skin to tan. This also happens when skin is exposed to other forms of ultraviolet light (such as in a tanning booth). If the skin receives too much ultraviolet light, the melanocytes suffer genetic damage and can be transformed into cancerous melanoma cells.

Because of the link to cumulative sun exposure, the chance of getting melanoma increases with age; however, many young people also get melanoma. In fact, melanoma is one of the most common cancers in young adults. Each year in the U.S., more than 50,000 people are diagnosed, and about 48,000 melanoma related deaths occur worldwide each year. The treatment includes surgical removal of the tumor, and if melanoma is found early while relatively small and thin, complete removal gives a high cure rate. The chance of the melanoma coming back or spreading depends on how deeply it has invaded into the layers of the skin. Thus, the more progressed the lesion, the great the chancer for recurrence and/or metastasis. For melanomas that recur or spread, treatments include chemo-plus immunotherapy or radiation therapy, but the prognosis for such patients, including those exhibiting metastatic disease (AJCC Stage III and IV) is poor, with 5-year survival rates being less than 10%. As such, improved treatments for melanoma, particularly for advanced metastatic disease, are urgently needed.

### SUMMARY

Thus, in accordance with the present disclosure, there is provided a method of inhibiting early metastasis in a subject having melanoma or breast cancer comprising administering to said subject an inhibitor of ALDH1L1, ALDH1L2 and MTHFD1. The subject may be a human or a non-human mammal. Inhibiting may comprise reducing the number metastatic lesions, inhibiting the growth of metastases, killing metastatic melanoma cells in lesions or in circulation, inducing remission, extending remission, inhibiting recurrence or inhibiting progression. The melanoma may AJCC late state II, stage III, or stage IV disease. The subject may have previously received a radiotherapy, a chemotherapy, an immunotherapy, a molecularly targeted therapy or had surgical resection of a tumor. The subject may have failed one or more standard melanoma therapies. The inhibitor may be an interfering RNA. Administering may comprise topical, intravenous, intraarterial, subcutaneous, oral or intra-tumoral administration. Administering may comprise local, regional or systemic administration, or may comprise continuous infusion over a period of time. The melanoma may or may not exhibit a pathogenic BRAF mutation. The pathogenic BRAF mutation may be V600E or V600K.

The method may further comprise administering a second anti-melanoma therapy, such as an anti-folate, a MEK inhibitor, a BRAF and/or a cardiac glycoside is administered before the other agents. One or more of the anti-folate, the MEK inhibitor, the BRAF inhibitor and/or the cardiac glycoside may be administered more than once. The cardiac glycoside may be selected from digoxin, digitoxin, gitoxin, oleandrin, neriifolin, bufalin, marinobufagenin, cinobufagenin, UNBS1450 and lanatoside C. The BRAF inhibitor may be vemurafenib, dabrafenib or sorafenib. The MEK inhibitor may be selected from trametinib, AS703026 (Pimasertib, MSC1936369B), AZD8330 (ARRY-424704), Selumetinib (AZD6244), PD-325901, GDC-0623, E6201, WX-554, R05126766, R04987655, AZD8330, Cometinib (XL518), Binimetinib, TAK-733 and CI-1040 (PD184352). The second anti-melanoma therapy may be a chemotherapeutic agent, such as dacarbazine. The second therapy anti-melanoma therapy may be given at the same time as said ALDH1L1/2 or MTHFD1 inhibitor, or given before, after or before and after said ALDH1L1/2 or MTHFD1 inhibitor. The anti-folate may be methotrexate or pemetrexed. The method may also further comprisie treating said subject with an immunomodulator, such as an antibody against PD1 or CTLA4, such as Ipilimumab. The immunomodulator may be imiquimod, IL-2 or interferon.

In another embodiment, there is provided a method of inhibiting melanoma early metastasis, inhibiting melanoma micrometastais, inhibiting melanoma progression and/or killing circulating melanoma cells in a subject having melanoma or breast cancer comprising administering to said subject an anti-folate. The subject may be a human or a non-human mammal. Inhibiting may comprise reducing the number metastatic lesions, inhibiting the growth of metastases, killing metastatic melanoma cells in lesions or in circulation, inducing remission, extending remission, inhibiting recurrence or inhibiting progression. The melanoma may AJCC late state II, stage III, or stage IV disease. The subject may have previously received a radiotherapy, a chemotherapy, an immunotherapy, a molecularly targeted therapy or had surgical resection of a tumor. The subject may have failed one or more standard melanoma therapies. The anti-folate therapy may be low dose methotrexate or pemetrexed, such as where low dose methotrexate is about 5 mg to about 20 mg oral dosage per week. Administering may comprise topical, intravenous, intraarterial, subcutaneous, oral or intra-tumoral administration. Administering may comprise local, regional or systemic administration, may comprise continuous infusion over a period of time. The melanoma may or may not exhibit a pathogenic BRAF mutation. The pathogenic BRAF mutation may be V600E or V600K.

The method may further comprise administering a second anti-melanoma therapy, such as a MEK inhibitor, a BRAF and/or a cardiac glycoside is administered before the other agents. One or more of the MEK inhibitor, the BRAF inhibitor and/or the cardiac glycoside may be administered more than once. The cardiac glycoside may be selected from digoxin, digitoxin, gitoxin, oleandrin, neriifolin, bufalin, marinobufagenin, cinobufagenin, UNBS1450 and lanatoside C. The BRAF inhibitor may be vemurafenib, dabrafenib or sorafenib. The MEK inhibitor may be selected from trametinib, AS703026 (Pimasertib, MSC1936369B), AZD8330 (ARRY-424704), Selumetinib (AZD6244), PD-325901, Cometinib (XL518), Binimetinib, TAK-733, GDC-0623, E6201, WX-554, RO5126766, RO4987655, AZD8330 and CI-1040 (PD184352). The second therapy anti-melanoma therapy is given at the same time as said anti-folate, or given before, after or before and after said anti-folate. The second anti-melanoma therapy may be a chemotherapeutic agent, such as dacarbazine. The method may further comprise treating said subject with an immunomodulator, such as an antibody against PD1 or CTLA4, such as Ipilimumab. The immunomodulator may be imiquimod, IL-2 or interferon.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-G. Multiple barriers to metastasis in vivo. (FIG. 1A) Live human melanoma cells (HLA+ and CD45/CD31/Ter119 negative) could be identified by flow cytometry in the blood of NSG mice bearing efficiently metastasizing melanomas. (FIG. 1B) Mice xenografted with efficiently metastasizing melanomas (n=43 mice with tumors derived from 4 patients) had significantly higher frequencies of circulating melanoma cells in their blood than mice xenografted with inefficiently metastasizing melanomas (n=13 mice with tumors derived from 4 patients) or control mice that had not been xenografted (n=18 mice). Blood was collected by cardiac puncture. Statistical significance was assessed using one-way analysis of variance (ANOVA) followed by Tukey's test for multiple comparisons (**, p<0.005). (FIGS. 1C-F) Bioluminescence analysis of total photon flux (photons/second) from mouse organs after intravenous injection (FIGS. 1C-D) or intrasplenic injection (FIGS. 1E-F) of luciferase-tagged melanoma cells derived from efficiently metastasizing (FIG. 1C, 1E) or inefficiently metastasizing (FIG. 1D, 1F) melanomas. Each melanoma was derived from a different patient (see FIGS. 6A and 6C) and was studied in an independent experiment. (FIG. 1G) Limiting dilution analysis of the frequency of tumor-forming cells after subcutaneous, intravenous, or intrasplenic transplantation into NSG mice. Statistical significance was assessed by a chi-square test using the ELDA software⁶³. (****, p<0.0001).

FIGS. 2A-E. Melanoma cells undergo reversible changes in tropism during metastasis. (FIG. 2A-B) Schematic (FIG. 2A) and limit dilution analysis (FIG. 2B) of the capacity of melanoma cells from primary subcutaneous tumors, the blood (circulating cells), and metastatic nodules to form tumors after subcutaneous, intravenous, and intrasplenic transplantation. These data reflect three independent experiments performed with efficiently metastasizing melanomas (M405, M481 and UT10; n=10 mice/melanoma/melanoma cell source/transplantation site for a total of 90 mice). (FIG. 2C-D) Schematic (FIG. 2C) and limit dilution analysis (FIG. 2D) of the capacity of melanoma cells from primary subcutaneous tumors, the blood (circulating cells), and metastatic nodules to form tumors after being passaged subcutaneously in primary recipient mice and then transplanted subcutaneously, intravenously, and intrasplenically into secondary recipient mice. These data reflect one experiment performed with efficiently metastasizing melanoma cells (M481; n=8-10 mice/melanoma cell source/transplantation site for a total of 85 mice). Statistical significance was assessed by a chi-square test using ELDA software⁶³ (*, p<0.05; **, p<0.005; ***, p<0.0005). (FIG. 2E) Summary of average limit dilution values in transplantation experiments showing reversible changes in the efficiency of tumor formation.

FIGS. 3A-G. Metastasizing melanoma cells experience high levels of oxidative stress. (FIG. 3A) GSH/GSSG ratio in primary subcutaneous tumors as compared to metastatic nodules (n=16 mice from 2 independent experiments with 4 different melanomas, M481, M405, M514, UT10). (FIG. 3B) GSH/GSSG ratio in primary subcutaneous tumors as compared to circulating melanoma cells (n=7 mice from 3 independent experiments with 2 different melanomas, M405 and UT10). (FIGS. 3C-D) cytoplasmic ROS levels (FIG. 3C) and mitochondrial ROS levels (FIG. 3D) in dissociated melanoma cells from primary subcutaneous tumors, the blood, and metastatic nodules obtained from the same mice (n=9 mice from 3 independent experiments using 3 different melanomas). (FIGS. 3E-F) Mitochondrial mass (FIG. 3E) and mitochondrial membrane potential (FIG. 3F) in dissociated melanoma cells from primary subcutaneous tumors, the blood, and metastatic nodules obtained from the same mice (n=6 mice from 2 independent experiments using 3 different melanomas). (FIG. 3G) Mitochondrial mass in dissociated melanoma cells from primary subcutaneous tumors versus metastatic nodules obtained from the same mice that had been transplanted with melanoma cells that originated from subcutaneous tumors (i), circulating cells (ii), or metastatic nodules (iii). These data indicate that melanoma cells undergo reversible changes in mitochondrial mass per cell during metastasis. All data represent mean±sd. Statistical significance was assessed using two-tailed Student's t-tests (FIGS. 3A-B) and one-way analyses of variance (ANOVAs) followed by Dunnett's tests for multiple comparisons (FIGS. 3C-G; *, p<0.05; ***, p<0.0005; ****, p<0.00005).

FIGS. 4A-I. Melanoma cell metastasis, but not primary subcutaneous tumor growth, is promoted by anti-oxidants and associated with increased flux through the folate pathway via de novo serine synthesis in vivo. (FIG. 4A) Growth of primary subcutaneous tumors in NSG mice treated with either PBS (Control) or N-acetyl-cysteine (NAC) daily by subcutaneous injection (n=10 mice/treatment for M405 and M481 and 5 mice/treatment for UT10 in three independent experiments). (FIG. 4B) Frequency of circulating melanoma cells in the blood of the mice from FIG. 4A. Metastatic disease burden assessed based on total bioluminescence signal from the visceral organs of the mice shown in FIG. 4A. (FIGS. 4D-G) In vivo isotope tracing of ¹³C-labelled glucose into 3-phosphoglycerate (FIG. 4G), lactate (FIG. 4E), serine (FIG. 4F), and glycine (FIG. 4G) in primary subcutaneous tumors versus metastatic nodules from the same mice (n=6 mice in 2 independent experiments for M405; n=3 mice in independent experiment each for M481 and UT10). (FIGS. 4H-I) Levels of NADPH (FIG. 4H) and NADP (FIG. 4I) in primary subcutaneous tumors versus metastatic nodules (n=4 mice from 2 independent experiments with M481 and UT10). All data represent mean±sd. Statistical significance was assessed using two-tailed Student's t-tests (FIGS. 4D-F, 4H and 4I), or Mann-Whitney test (FIG. 4G), due to unequal variance after transformation of the data, and repeated-measures two-way analyses of variance (ANOVAs) (FIGS. 4A-C; *. p<0.05; **, p<0.005; ***, p<0.0005, ****, p<0.00005).

FIGS. 5A-K. During metastasis, some melanoma cells reversibly increase their expression of folate pathway enzymes that generate NADPH and folate pathway inhibition selectively impairs metastasis. (FIG. 5A) Western blot analysis of folate pathway enzymes in subcutaneous tumours versus metastatic liver and kidney nodules from NSG mice transplanted with three different melanomas. (FIGS. 5B-D) Growth of subcutaneous tumors in mice bearing three different melanomas (M405, M481, UT10) treated with DMSO (control) or methotrexate (n=5 mice/treatment). The frequency of circulating melanoma cells in the blood (FIG. 5C) and metastatic disease burden (FIG. 5D) in the same mice (n=10 mice/treatment for each melanoma except n=8 for M405). Data in FIGS. 5B-D reflect 6 independent experiments but only one representative experiment per melanoma is shown in FIG. 5B. (FIGS. 5E-G) Growth of subcutaneous tumours in mice transplanted with two different melanomas expressing scrambled control shRNA versus two shRNAs against ALDH1L2. The frequency of circulating melanoma cells (FIG. 5F) and metastatic disease burden in visceral organs based on total bioluminescence signal (FIG. 5G). The data in FIGS. 5E-G reflect 6 independent experiments (n=10 mice per shRNA for M405 and n=19 mice per shRNA for M481) but only one representative experiment per melanoma is shown in panel (FIGS. 5D-H) Western blot analysis of ALDH1L2 expression in subcutaneous tumours versus metastatic liver nodules from a donor mouse or from recipient mice subcutaneously transplanted with subcutaneous, circulating, or metastatic melanoma cells from the donor mouse. The increase in ALDH1L2 expression in metastatic liver nodules was reversible upon subcutaneous transplantation. Data in FIGS. 5A and 5H are from two independent experiments. (FIGS. 5I-K) Growth of subcutaneous tumours in mice transplanted with cells from two melanomas expressing either scrambled control shRNAs or two shRNAs against MTFHD1. Frequency of circulating melanoma cells (FIG. 5J) and metastatic disease burden in visceral organs (FIG. 5K) from the same mice. Data in FIGS. 5I-K reflect four independent experiments with a total of 9 mice per control shRNA and 10 mice per shRNA against MTFHD1 for each melanoma. Panel i shows one representative experiment per melanoma. All error bars represent standard deviation. Statistical significance was assessed using one-way analyses of variance (ANOVAs) followed by Dunnett's tests for multiple comparisons (FIGS. 5F, 5G, 5J and 5K), two-tailed Student's t-tests (FIGS. 5C and 5D) and repeated measures two-way ANOVAs (FIGS. 5B, 5E and 50 (*, p<0.05; **, p,0.005, ***, p<0.0005; ****, p<0.00005).

FIGS. 6A-C. Clinical data on the melanomas used in this study and summary of their metastatic behavior in NSG mice. (FIG. 6A) Summary of the clinical characteristics of the melanomas used in this study at the time of banking, as well as patient outcome after banking, and metastasis patterns upon transplantation of banked tumors into NSG mice. Melanomas were stratified into efficient and inefficient metastasizers. Efficient metastasiers formed distant metastases in patients and in NSG mice. Inefficient metastasizers did not form distant metastases in patients or distant macrometastases in NSG mice, or micrometastases outside of the lung. (FIG. 6B) Growth rates of primary subcutaneous tumors in NSG mice after subcutaneous transplantation of 100 cells. Statistical significance was assessed using two-tailed Student's t-test. (FIG. 6C) Clinical characteristics of the patients from whom melanomas were obtained at the time of banking and upon subsequent clinical follow up.

FIG. 7. Unsupervised clustering of metabolomic analysis of primary subcutaneous tumors versus metastatic nodules obtained from NSG mice. Hierarchical clustering using Pearson distance and complete linkage of metabolites from five different melanomas (n=2-3 mice/melanoma in two independent experiments).

### DETAILED DESCRIPTION

Circulating cancer cells are commonly observed in the blood of patients and mice with various cancers (Yu et al., 2013, Stott et al., 2010, Yu et al., 2014 and Nagrath et al., 2007). However, metastasis is a very inefficient process (Vanharanta & Massague 2013) in which few disseminated cancer cells survive after extravasation from the blood into distant organs and even fewer proliferate to form micrometastases (Luzzi et al., 1998, Cameron et al., 2000 and Kienast et al., 2010). Circulating cancer cells in patients are associated with worse outcomes in some contexts (Joosse et al., 2014); however, some patients can have circulating cancer cells in their blood without any evidence of metastasis or worse outcomes (Engell, 1959, Griffiths et al., 1973 and Salsbury et al., 1975). These studies raise the fundamental question of why circulating cancer cells rarely give rise to metastases.

Epithelial cells undergo cell death when they detach from extracellular matrix in culture as a result of reduced glucose uptake, ATP depletion, and oxidative stress (Debnath & Brugge, 2005 and Debnath et al., 2002). However, oncogenic signaling can promote the survival of detached breast epithelial cells by increasing glucose uptake and flux through the pentose phosphate pathway, which generates NADPH and regenerates glutathione, a buffer against oxidative stress (Schafer et al., 2009). Cancer cells thus undergo genetic changes within primary tumors that increase their capacity to withstand oxidative stress, raising the question of whether additional adaptations are required during metastasis. Breast cancer cell lines undergo metabolic changes during invasion in culture and metastasis in vivo that would be expected to reduce the generation of reactive oxygen species (ROS) (Dong et al., 2013, Kamarajugadda et al., 2013, Qu et al., 2011, Chen et al., 2007 and Lu et al., 2010). Nonetheless, to the inventors' knowledge it is unknown whether ROS levels change in metastasizing cells in vivo or whether this limits distant metastasis. In fact, studies of other cell lines, including melanoma cell lines, have found that anti-oxidants inhibit metastasis, raising the possibility that ROS actually promote metastasis (Porporato et al., 2014, LeBleu et al., 2014 and Ishikawa et al., 2008).

The inventors have addressed these issues by studying melanomas from multiple patients that were xenografted into NOD/SCID IL2Rγ^{null} (NSG) mice. Melanoma metastasis in this assay is predictive of clinical outcome in patients: stage III melanomas that metastasize efficiently in NSG mice go on to form distant metastases in patients whereas melanomas that metastasize inefficiently in mice do not form distant metastases in patients (Quintana et al., 2012). They used this in vivo assay to compare the ability of efficiently metastasizing and inefficiently metastasizing human melanomas to form tumors upon subcutaneous versus intravenous versus intrasplenic injection. Melanoma cells much more readily formed tumors upon subcutaneous as compared to intravenous or intrasplenic injection, where they experienced high levels of oxidative stress. Efficient metastasizers formed tumors more readily than inefficient metastasizers upon intravenous or intrasplenic injection by undergoing reversible metabolic changes that increased their ability to cope with oxidative stress. These results suggest that oxidative stress may represent a general barrier to distant metastasis by cancer cells in vivo. These and other aspects of the disclosure are described in detail below.

### I. FOLATE PATHWAY

Folic acid or folate is a B vitamin. It is also referred to as vitamin M, vitamin B₉, vitamin B_{c} (or folacin), pteroyl-L-glutamic acid, and pteroyl-L-glutamate. Food supplement manufacturers often use the term folate for something different than “pure” folic acid: in chemistry, folate refers to the deprotonated ion, and folic acid to the neutral molecule—which both co-exist in water. The food industry often uses folate to mean the natural occurring form of folic acid. There is no “natural” and chemical form of folate and folic acid, and the two terms can be used interchangeably. It is likely that in the food industry, folate indicates a collection of “folates” that is not chemically well-characterized, including other members of the family of pteroylglutamates, or mixtures of them, having various levels of reduction of the pteridine ring, one-carbon substitutions and different numbers of glutamate residues, all of which occur in nature.

Folic acid is synthetically produced, and used in fortified foods and supplements. Folate is converted by humans to dihydrofolate (dihydrofolic acid), tetrahydrofolate (tetrahydrofolic acid), and other derivatives, which have various biological activities. Vitamin B₉ is essential for numerous bodily functions. Humans cannot synthesize folates de novo; therefore, folic acid has to be supplied through the diet to meet their daily requirements. The human body needs folate to synthesize DNA, repair DNA, and methylate DNA as well as to act as a cofactor in certain biological reactions. It is especially important in aiding rapid cell division and growth, such as in infancy and pregnancy. Children and adults both require folate to produce healthy red blood cells and prevent anemia. Folates occur naturally in many foods and, among plants, are especially plentiful in dark green leafy vegetables.

A lack of dietary folates can lead to folate deficiency. A complete lack of dietary folate takes months before deficiency develops as normal individuals have about 500-20,000 μg of folate in body stores. This deficiency can result in many health problems, the most notable one being neural tube defects in developing embryos—a relatively rare birth defect affecting only 300,000 (0.002%) of births globally each year. Common symptoms of folate deficiency include diarrhea, macrocytic anemia with weakness or shortness of breath, nerve damage with weakness and limb numbness (peripheral neuropathy), pregnancy complications, mental confusion, forgetfulness or other cognitive deficits, mental depression, sore or swollen tongue, peptic or mouth ulcers, headaches, heart palpitations, irritability, and behavioral disorders. Low levels of folate can also lead to homocysteine accumulation. Low levels of folate have been associated with specific cancers. However, it is not clear whether consuming recommended (or higher) amounts of folic acid—from foods or in supplements—can lower cancer risk in some people.

Diets high in folate are associated with decreased risk of colorectal cancer; some studies show the association is stronger for folate from foods alone than for folate from foods and supplements, One broad cancer screening trial reported a potential harmful effect of too much folate intake on breast cancer risk, suggesting routine folate supplementation should not be recommended as a breast cancer preventive. Most research studies indicate that dietary folate intake does not significantly increase or decrease the risk of prostate cancer.

Folate is important for cells and tissues that rapidly divide. Cancer cells divide rapidly, and drugs that interfere with folate metabolism are used to treat cancer. The antifolate methotrexate is a drug often used to treat cancer because it inhibits the production of the active form of THF from the inactive dihydrofolate (DHF). However, methotrexate can be toxic, producing side effects, such as inflammation in the digestive tract that make it difficult to eat normally. Also, bone marrow depression (inducing leukopenia and thrombocytopenia), and acute kidney and liver failure have been reported. Folic acid supplementation does not appear to affect the rate of cancer.

Folinic acid, under the drug name leucovorin, a form of folate (formyl-THF), can help “rescue” or reverse the toxic effects of methotrexate. Folinic acid is not the same as folic acid. Folic acid supplements have little established role in cancer chemotherapy. There have been cases of severe adverse effects of accidental substitution of folic acid for folinic acid in patients receiving methotrexate cancer chemotherapy. It is important for anyone receiving methotrexate to follow medical advice on the use of folic or folinic acid supplements. The supplement of folinic acid in patients undergoing methotrexate treatment is to give cells dividing less rapidly enough folate to maintain normal cell functions. The amount of folate given is depleted by rapidly dividing cells (cancer) quickly, and so does not negate the effects of methotrexate.

### II. THERAPEUTIC AGENTS

A. Anti-Folate

In accordance with the present disclosure, the inventors propose the use of anti-folate agents for the inhibition of metastatic melanoma. Antifolates are drugs that antagonise (that is, block) the actions of folic acid (vitamin B₉). Folic acid's primary function in the body is as a cofactor to various methyltransferases involved in serine, methionine, thymidine and purine biosynthesis. Consequently antifolates inhibit cell division, DNA/RNA synthesis and repair and protein synthesis. Some such as proguanil, pyrimethamine and trimethoprim selectively inhibit folate's actions in microbial organisms such as bacteria, protozoa and fungi. The majority of antifolates work by inhibiting dihydrofolate reductase (DHFR), but raltitrexed is an inhibitor of thymidylate synthase, and pemetrexed inhibits both and a third enzyme. Antifolates act specifically during DNA and RNA synthesis, and thus are cytotoxic during the S-phase of the cell cycle. Thus, they have a greater toxic effect on rapidly dividing cells (such as malignant and myeloid cells, and GI & oral mucosa), which replicate their DNA more frequently, and thus inhibits the growth and proliferation of these non-cancerous cells as well as causing certain side-effects.

Methotrexate, abbreviated MTX and formerly known as amethopterin, is an antimetabolite and antifolate drug. It is used in treatment of cancer, autoimmune diseases, ectopic pregnancy, and for the induction of medical abortions. It acts by inhibiting the metabolism of folic acid. Methotrexate began to replace the more toxic antifolate aminopterin starting in the 1950s. Methotrexate was originally developed and continues to be used for chemotherapy, either alone or in combination with other agents. It is effective for the treatment of a number of cancers including: breast, head and neck, leukemia, lymphoma, lung, osteosarcoma, bladder, and trophoblastic neoplasms. Methotrexate can be taken orally or administered by injection (intramuscular, intravenous, subcutaneous, or intrathecal). Oral doses are taken weekly, not daily, to limit toxicity. Routine monitoring of the complete blood count, liver function tests, and creatinine are recommended. Measurements of creatinine are recommended at least every 2 months.

The most common adverse effects include hepatotoxicity (liver damage), ulcerative stomatitis, low white blood cell count and thus predisposition to infection, nausea, abdominal pain, fatigue, fever, dizziness, acute pneumonitis, rarely pulmonary fibrosis and kidney failure. Methotrexate is teratogenic (harmful to fetus) and hence not used in pregnancy (pregnancy category X). Central nervous system reactions to methotrexate have been reported, especially when given via the intrathecal route, which include myelopathies and leucoencephalopathies. It has a variety of cutaneous side effects, particularly when administered in high doses. Another little understood but serious possible adverse effect of methotrexate is Neurological damage and memory loss. Neurotoxicity may result from the drug crossing the blood-brain barrier and damaging neurons in the cerebral cortex. Cancer patients who receive the drug often nickname these effects ‘Chemo brain or ‘Chemo fog’.

Penicillins may decrease the elimination of methotrexate and thus increase the risk of toxicity. While they may be used together increased monitoring is recommended. The aminoglycosides, neomycin and paromomycin, have been found to reduce GI absorption of methotrexate. Probenecid inhibits methotrexate excretion, which increases the risk of methotrexate toxicity. Likewise retinoids and trimethoprim have been known to interact with methotrexate to produce additive hepatotoxicity and haematotoxicity, respectively. Other immunosuppressants like ciclosporin may potentiate methotrexate's haematologic effects, hence potentially leading to toxicity. NSAIDs have also been found to fatally interact with methotrexate in numerous case reports. Nitrous oxide potentiating the haematological toxicity of methotrexate has also been documented. Proton-pump inhibitors like omeprazole and the anticonvulsant valproate have been found to increase the plasma concentrations of methotrexate, as have nephrotoxic agents such as cisplatin, the GI drug, colestyramine and dantrolene. Caffeine may antagonise the effects methotrexate on rheumatoid arthritis by antagonising the receptors for adenosine.

Methotrexate is thought to affect cancer and rheumatoid arthritis by two different pathways. For cancer, methotrexate competitively inhibits dihydrofolate reductase (DHFR), an enzyme that participates in the tetrahydrofolate synthesis. The affinity of methotrexate for DHFR is about one thousand-fold that of folate. DHFR catalyses the conversion of dihydrofolate to the active tetrahydrofolate. Folic acid is needed for the de novo synthesis of the nucleoside thymidine, required for DNA synthesis. Also, folate is essential for purine and pyrimidine base biosynthesis, so synthesis will be inhibited. Methotrexate, therefore, inhibits the synthesis of DNA, RNA, thymidylates, and proteins.

For the treatment of rheumatoid arthritis, inhibition of DHFR is not thought to be the main mechanism, but rather multiple mechanisms appear to be involved including: the inhibition of enzymes involved in purine metabolism, leading to accumulation of adenosine; inhibition of T cell activation and suppression of intercellular adhesion molecule expression by T cells; selective down-regulation of B cells; increasing CD95 sensitivity of activated T cells; inhibition of methyltransferase activity, leading to (de)-activation of enzyme activity relevant to immune system function. Another mechanism of MTX is the inhibition of the binding of Interleukin 1 beta to its cell surface receptor.

Pemetrexed (brand name Alimta®) is a chemotherapy drug manufactured and marketed by Eli Lilly and Company. Its indications are the treatment of pleural mesothelioma and non-small cell lung cancer. Pemetrexed is chemically similar to folic acid and is in the class of chemotherapy drugs called folate antimetabolites. It works by inhibiting three enzymes used in purine and pyrimidine synthesis—thymidylate synthase (TS), dihydrofolate reductase (DHFR), and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

In February 2004, the Food and Drug Administration approved pemetrexed for treatment of malignant Pleural Mesothelioma, a type of tumor of the lining of the lung, in combination with cisplatin for patients whose disease is either unresectable or who are not otherwise candidates for curative surgery. In September 2008, the FDA granted approval as a first-line treatment, in combination with cisplatin, against locally-advanced and metastatic non-small cell lung cancer (NSCLC) in patients with non-squamous histology. A Phase III study showed benefits of maintenance use of pemetrexed for non-squamous NSCLC. Activity has been shown in malignant peritoneal mesothelioma. Trials are currently testing it against esophagus and other cancers.

Pemetrexed is also recommended in combination with carboplatin for the first-line treatment of advanced non-small cell lung cancer. However, the relative efficacy or toxicity of pemetrexed-cisplatin versus pemetrexed-carboplatin has not been established beyond what is generally thought about cisplatin or carboplatin doublet drug therapy.

Other anti-folates include proguanil, pyrimethamine, and trimethoprin, which are specifically contempated as agents as well.

B. ALDH1L Inhibitors

1. Background

Aldehyde dehydrogenases are a group of enzymes that catalyse the oxidation (dehydrogenation) of aldehydes. To date, nineteen ALDH genes have been identified within the human genome. These genes participate in a wide variety of biological processes including the detoxification of exogenously and endogenously generated aldehydes.

Aldehyde dehydrogenase is a polymorphic enzyme responsible for the oxidation of aldehydes to carboxylic acids, which leave the liver and are metabolized by the body's muscle and heart. There are three different classes of these enzymes in mammals: class 1 (low Kₘ, cytosolic), class 2 (low Kₘ, mitochondrial), and class 3 (high Kₘ, such as those expressed in tumors, stomach, and cornea). In all three classes, constitutive and inducible forms exist. ALDH1 and ALDH2 are the most important enzymes for aldehyde oxidation, and both are tetrameric enzymes composed of 54 kD subunits. These enzymes are found in many tissues of the body but are at the highest concentration in the liver.

The active site of the aldehyde dehydrogenase enzyme is largely conserved throughout the different classes of the enzyme and, although the number of amino acids present in a subunit can change, the overall function of the site changes little. The active site binds to one molecule of an aldehyde and an NAD(P)⁺ that functions as a cofactor. A cysteine and a glutamate will interact with the aldehyde substrate. Many other residues will interact with the NAD(P)⁺ to hold it in place. A magnesium may be used to help the enzyme function, although the amount it helps the enzyme can vary between different classes of aldehydes.

ALDH2 plays a crucial role in maintaining low blood levels of acetaldehyde during alcohol oxidation. In this pathway, the intermediate structures can be toxic, and health problems arise when those intermediates cannot be cleared. When high levels of acetaldehyde occur in the blood, facial flushing, light headedness, palpitations, nausea, and general “hangover” symptoms occur. These symptoms are indicative of a disease known as the Alcohol flush reaction, also known as “Asian Flush” or “Oriental Flushing Syndrome.”

There is a mutant form of aldehyde dehydrogenase, termed ALDH2*2, wherein a lysine residue replaces a glutamate in the active site at position 487 of ALDH2. Homozygous individuals with the mutant allele have almost no ALDH2 activity, and those heterozygous for the mutation have reduced activity. Thus, the mutation is partially dominant. The ineffective homozygous allele works at a rate of about 8% of the normal allele, for it shows a higher km for NAD⁺ and has a higher maximum velocity than the wild-type allele. This mutation is common in Japan, where 41% of a non-alcoholic control group were ALDH2 deficient, where only 2-5% of an alcoholic group were ALDH2-deficient. In Taiwan, the numbers are similar, with 30% of the control group showing the deficiency and 6% of alcoholics displaying it. The deficiency is manifested by slow acetaldehyde removal, with low alcohol tolerance perhaps leading to a lower frequency of alcoholism.

These symptoms are the same as those observed in people who drink while being treated by the drug disulfiram, which is why it is used to treat alcoholism. The patients show higher blood levels of acetaldehyde, and become violently ill upon consumption of even small amounts of alcohol. Several drugs (e.g., metronidazole) cause a similar reaction known as “disulfiram-like reaction.”

Yokoyama et al. found that decreased enzyme activity of aldehyde dehydrogenase-2, caused by the mutated ALDH2 allele, contributes to a higher chance of esophageal and oropharyngolaryngeal cancers. The metabolized acetaldehyde in the blood, which is six times higher than in individuals without the mutation, has shown to be a carcinogen in lab animals. ALDH2*2 is associated with increased odds of oropharyngolaryngeal, esophageal, gastric, colon, and lung cancer. However, they found no connection between increased levels of ALDH2*2 in the blood and an increased risk of liver cancer.

Fitzmaurice et al. explored Aldehyde dehydrogenase inhibition as a pathogenic mechanism in Parkinson disease. “This ALDH model for PD etiology may help explain the selective vulnerability of dopaminergic neurons in PD and provide a potential mechanism through which environmental toxicants contribute to PD pathogenesis.”

ALDH1L1.

10-formyltetrahydrofolate dehydrogenase (ALDH1L1) is an enzyme that in humans is encoded by the ALDH1L1 gene. The ALDH1L1 protein catalyzes the conversion of 10-formyltetrahydrofolate, NADP, and water to tetrahydrofolate, NADPH, and carbon dioxide. The encoded protein belongs to the aldehyde dehydrogenase family and is responsible for formate oxidation in vivo. Deficiencies in this gene can result in an accumulation of formate and subsequent methanol poisoning. The accession nos. for the human mRNA and protein, respectively, are NM_001270364 and NP_001257293.

ALDH1L2.

Aldehyde dehydrogenase 1 family, member L2, also known as ALDH1L2, is an enzyme that in humans is encoded by the ALDH1L2 gene. ALDH1L2 is the mitochondrial isoform of a similar enzyme, ALDH1L1, which converts 10-formyltetrahydrofolate to tetrahydrofolate and carbon dioxide. The accession nos. for the human mRNA and protein, respectively, are NM_001034173 and NP_001029345.

MTHFD1.

Methylenetetrahydrofolate Dehydrogenase (NADP+ Dependent) 1, also known as MTHFD1, in an enzyme that in humans is encoded by the MTHFD1 gene. MTHFD1 is the cytoplasmic enzyme, that has three distinct enzymatic activities, 5,10-methylenetetrahydrofolate dehydrogenase, 5,10-methenyltetrahydrofolate cyclohydrolase and 10-formyltetrahydrofolate synthetase. Each of these activities catalyzes one of three sequential reactions in the interconversion of 1-carbon derivatives of tetrahydrofolate, which are substrates for methionine, thymidylate, and de novo purine syntheses.

2. Inhibitors

Inhibitors of ALDH1L1/2 and MTHFD1 will find use in the therapeutic methods described below. The inhibitor may be a protein, a nucleic acid or a small molecule. Protein inhibitors and nucleic acid inhibitors are described in some detail below.

i. Protein Inhibitors

In one embodiment, the inhibitor of ALDH1L1/2 and MTHFD1 may be a proteinacenous inhibitor. Proteinaceous inhibitors generally fall into two categories—antibodies that bind to the enzyme, in this case ALDH1L1/2 and MTHFD1, or protein fragments of the target that retain to ability to bind the substrate, but fail to enzymatically process the same.

Antibodies according to the present disclosure may be defined, in the first instance, by their binding specificity, which in this case is for ALDH1L1/2 and MTHFD1. Those of skill in the art, by assessing the binding specificity/affinity of a given antibody using techniques well known to those of skill in the art, can determine whether such antibodies fall within the scope of the instant claims.

In various embodiments, one may choose to engineer sequences of the identified antibodies for a variety of reasons, such as improved expression, improved cross-reactivity or diminished off-target binding.

Antibody molecules also comprise antibody fragments (such as F(ab′), F(ab′)2) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The present disclosure also contemplates isotype modification. By modifying the Fc region to have a different isotype, different functionalities can be achieved. For example, changing to IgGi can increase antibody dependent cell cytotoxicity, switching to class A can improve tissue distribution, and switching to class M can improve valency.

Modified antibodies may be made by any technique known to those of skill in the art, including expression through standard molecular biological techniques, or the chemical synthesis of polypeptides. Methods for recombinant expression are addressed elsewhere in this document.

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage diplay and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

ii. Nucleic Acid Inhibitors

In another embodiment, the inhibitor of ALDH1L1/2 may be a nucleic acid inhibitor. Such inhibitors include antisense molecules, ribozyme and inhibitory oligonucleotides, often referred to as interfering RNAs (e.g., siRNAs, shRNAs, miRNAs). The latter rely on RNA interference (RNAi), a biological process in which RNA molecules inhibit gene expression, typically by causing the destruction of specific mRNA molecules. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from producing a protein. RNA interference has an important role in defending cells against parasitic nucleotide sequences—viruses and transposons. It also influences development.

The RNAi pathway is found in many eukaryotes, including animals, and is initiated by the enzyme Dicer, which cleaves long double-stranded RNA (dsRNA) molecules into short double stranded fragments of ˜20 nucleotide siRNAs. Each siRNA is unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in a messenger RNA molecule and induces cleavage by Argonaute, the catalytic component of the RISC complex. In some organisms, this process spreads systemically, despite the initially limited molar concentrations of siRNA.

siRNAs.

Small interfering RNA (siRNA), sometimes known as short interfering RNA or silencing RNA, is a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation. siRNA also acts in RNAi-related pathways, e.g., as an antiviral mechanism or in shaping the chromatin structure of a genome. The complexity of these pathways is only now being elucidated. siRNAs and their role in post-transcriptional gene silencing (PTGS) in plants were first discovered by David Baulcombe's group in 1999. Thomas Tuschl and colleagues soon reported that synthetic siRNAs could induce RNAi in mammalian cells. siRNAs have a well-defined structure: a short (usually 20 to 24-bp) double-stranded

RNA (dsRNA) with phosphorylated 5′ ends and hydroxylated 3′ ends with two overhanging nucleotides. The Dicer enzyme catalyzes production of siRNAs from long dsRNAs and small hairpin RNAs. siRNAs can also be introduced into cells by transfection. Since in principle any gene can be knocked down by a synthetic siRNA with a complementary sequence, siRNAs are an important tool for validating gene function and drug targeting in the post-genomic era.

shRNAs.

A small hairpin RNA or short hairpin RNA (shRNA) is an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover. However, it requires use of an expression vector, which can pose safety concerns.

The promoter choice is essential to achieve robust shRNA expression. At first, polymerase III promoters such as U6 and H1 were used; however, these promoters lack spatial and temporal control. As such, there has been a shift to using polymerase II promoters to regulate shRNA expression.

Expression of shRNA in cells can be obtained by delivery of plasmids or through viral or bacterial vectors. Delivery of plasmids to cells through transfection to obtain shRNA expression can be accomplished using commercially available reagents in vitro. However, this method is not applicable in vivo and thus has limited utility.

Use of a bacterial vector to obtain shRNA expression in cells is a relatively recent approach. It builds off research showing that recombinant Escherichia coli, containing a plasmid with shRNA, fed to mice can knock-down target gene expression in the intestinal epithelium.

A variety of viral vectors can be used to obtain shRNA expression in cells including adeno-associated viruses (AAVs), adenoviruses, and lentiviruses. With adeno-associated viruses and adenoviruses, the genomes remain episomal. This is advantageous as insertional mutagenesis is avoided. It is disadvantageous in that the progeny of the cell will lose the virus quickly through cell division unless the cell divides very slowly. AAVs differ from adenoviruses in that the viral genes have been removed and they have diminished packing capacity. Lentiviruses integrate into sections of transcriptionally active chromatin and are thus passed on to progeny cells. With this approach there is increased risk of insertional mutagenesis; however, the risk can be reduced by using an integrase-deficient lentivirus.

Once the vector has integrated into the host genome, the shRNA is then transcribed in the nucleus by polymerase II or polymerase III depending on the promoter choice. This product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing.

miRNAs.

A microRNA (abbreviated miRNA) is a small non-coding RNA molecule (containing about 22 nucleotides) found in plants, animals, and some viruses, which functions in RNA silencing and post-transcriptional regulation of gene expression. Encoded by eukaryotic nuclear DNA in plants and animals and by viral DNA in certain viruses whose genome is based on DNA, miRNAs function via base-pairing with complementary sequences within mRNA molecules. As a result, these mRNA molecules are silenced by one or more of the following processes: 1) cleavage of the mRNA strand into two pieces, 2) destabilization of the mRNA through shortening of its poly(A) tail, and 3) less efficient translation of the mRNA into proteins by ribosomes. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. The human genome may encode over 1000 miRNAs, which are abundant in many mammalian cell types and appear to target about 60% of the genes of humans and other mammals.

miRNAs are well conserved in both plants and animals, and are thought to be a vital and evolutionarily ancient component of genetic regulation. While core components of the microRNA pathway are conserved between plants and animals, miRNA repertoires in the two kingdoms appear to have emerged independently with different primary modes of action. Plant miRNAs usually have near-perfect pairing with their mRNA targets, which induces gene repression through cleavage of the target transcripts. In contrast, animal miRNAs are able to recognize their target mRNAs by using as little as 6-8 nucleotides (the seed region) at the 5′ end of the miRNA, which is not enough pairing to induce cleavage of the target mRNAs. Combinatorial regulation is a feature of miRNA regulation in animals. A given miRNA may have hundreds of different mRNA targets, and a given target might be regulated by multiple miRNAs.

The first miRNA was discovered in the early 1990s. However, miRNAs were not recognized as a distinct class of biological regulators until the early 2000s. Since then, miRNA research has revealed different sets of miRNAs expressed in different cell types and tissues and has revealed multiple roles for miRNAs in plant and animal development and in many other biological processes. Aberrant expression of miRNAs has been implicated in numerous disease states, and miRNA-based therapies are under investigation.

Estimates of the average number of unique messenger RNAs that are targets for repression by a typical microRNA vary, depending on the method used to make the estimate, but several approaches show that mammalian miRNAs can have many unique targets. For example, an analysis of the miRNAs highly conserved in vertebrate animals shows that each of these miRNAs has, on average, roughly 400 conserved targets. Likewise, experiments show that a single miRNA can reduce the stability of hundreds of unique messenger RNAs, and other experiments show that a single miRNA may repress the production of hundreds of proteins, but that this repression often is relatively mild (less than 2-fold).

### III. METHODS OF TREATMENT

In a particular aspect, the present disclosure provides methods for the treatment of metastatic melanoma and breast cancer. Treatment methods will involve administering to an individual having such a disease an effective amount of a composition containing the compounds of the present disclosure. An effective amount is described, generally, as that amount sufficient to detectably and repeatedly to ameliorate, reduce, minimize or limit the extent of metastasis. More specifically, it is envisioned that the treatment with compounds of the present disclosure kill metastatic cancer cells, inhibit their dissemination, reduce or inhibit the extent of metastatic lesions, inhibit or reduce or delay recurrence, or otherwise provide clinical benefit. Also, combinations my reduce toxicity due to lower dosing and or reduced frequency of administration.

A. Melanoma

Melanoma is less common than other skin cancers. However, it is much more dangerous if it is not found early. It causes the majority (75%) of deaths related to skin cancer. Worldwide, doctors diagnose about 160,000 new cases of melanoma yearly. It is more common in women than in men. In women, the most common site is the legs and melanomas in men are most common on the back. It is particularly common among Caucasians, especially northern Europeans living in sunny climates. There are high rates of incidence in Australia, New Zealand, North America (especially Texas and Florida), Latin America, and Northern Europe, with a paradoxical decrease in southern Italy and Sicily. This geographic pattern reflects the primary cause, ultraviolet light (UV) exposure crossed with the amount of skin pigmentation in the population.

1. Early Signs

Early signs of melanoma are changes to the shape or color of existing moles or, in the case of nodular melanoma, the appearance of a new lump anywhere on the skin (such lesions should be referred without delay to a dermatologist). At later stages, the mole may itch, ulcerate or bleed. Early signs of melanoma are summarized by the mnemonic “ABCDE”:

Asymmetry

Borders (irregular)

Color (variegated)

Diameter (greater than 6 mm (0.24 in), about the size of a pencil eraser)

Evolving over time

These classifications do not, however, apply to the most dangerous form of melanoma, nodular melanoma, which has its own classifications:

Elevated above the skin surface

Firm to the touch

Growing

Metastatic melanoma may cause nonspecific paraneoplastic symptoms, including loss of appetite, nausea, vomiting and fatigue. Metastasis of early melanoma is possible, but relatively rare: less than a fifth of melanomas diagnosed early become metastatic. Brain metastases are particularly common in patients with metastatic melanoma. It can also spread to the liver, bones, abdomen or distant lymph nodes.

2. Development

The earliest stage of melanoma starts when the melanocytes begin to grow out of control. Melanocytes are found between the outer layer of the skin (the epidermis) and the next layer (the dermis). This early stage of the disease is called the radial growth phase, and the tumor is less than 1 mm thick. Because the cancer cells have not yet reached the blood vessels lower down in the skin, it is very unlikely that this early-stage cancer will spread to other parts of the body. If the melanoma is detected at this stage, then it can usually be completely removed with surgery. When the tumor cells start to move in a different direction—vertically up into the epidermis and into the papillary dermis—the behavior of the cells changes dramatically.

The next step in the evolution is the invasive radial growth phase, which is a confusing term; however, it explains the next step in the process of the radial growth, when individual cells start to acquire invasive potential. This step is important—from this point on the melanoma is capable of spreading. The Breslow's depth of the lesion is usually less than 1 mm (0.04 in), the Clark level is usually 2.

The following step in the process is the invasive melanoma—the vertical growth phase (VGP). The tumor attains invasive potential, meaning it can grow into the surrounding tissue and can spread around the body through blood or lymph vessels. The tumor thickness is usually more than 1 mm (0.04 in), and the tumor involves the deeper parts of the dermis. The host elicits an immunological reaction against the tumor (during the VGP), which is judged by the presence and activity of the tumor infiltrating lymphocytes (TILs). These cells sometimes completely destroy the primary tumor; this is called regression, which is the latest stage of the melanoma development. In certain cases, the primary tumor is completely destroyed and only the metastatic tumor is discovered.

3. Detection

Visual diagnosis of melanomas is still the most common method employed by health professionals. Moles that are irregular in color or shape are often treated as candidates of melanoma. The diagnosis of melanoma requires experience, as early stages may look identical to harmless moles or not have any color at all. People with a personal or family history of skin cancer or of dysplastic nevus syndrome (multiple atypical moles) should see a dermatologist at least once a year to be sure they are not developing melanoma. There is no blood test for detecting melanomas. Metastatic melanomas can be detected by X-rays, CT scans, MRIs, PET and PET/CTs, ultrasound, LDH testing and photoacoustic detection.

Many melanomas present themselves as lesions smaller than 6 mm in diameter; and all melanomas were malignant on day 1 of growth, which is merely a dot. An astute physician will examine all abnormal moles, including ones less than 6 mm in diameter. Seborrheic keratosis may meet some or all of the ABCD criteria, and can lead to false alarms among laypeople and sometimes even physicians. An experienced doctor can generally distinguish seborrheic keratosis from melanoma upon examination, or with dermatoscopy.

Total body photography, which involves photographic documentation of as much body surface as possible, is often used during follow-up of high-risk patients. The technique has been reported to enable early detection and provides a cost-effective approach (being possible with the use of any digital camera), but its efficacy has been questioned due to its inability to detect macroscopic changes. The diagnosis method should be used in conjunction with (and not as a replacement for) dermoscopic imaging, with a combination of both methods appearing to give extremely high rates of detection.

Melanoma is divided into the following types:
Lentigo maligna
Lentigo maligna melanoma
Superficial spreading melanoma
Acral lentiginous melanoma
Mucosal melanoma
Nodular melanoma
Polypoid melanoma
Desmoplastic melanoma
Amelanotic melanoma
Soft-tissue melanoma
Melanoma with small nevus-like cells
Melanoma with features of a Spitz nevus
Uveal melanoma
   Confirmation of the clinical diagnosis is achieved with a skin biopsy. This is usually followed up with a wider excision of the scar or tumor. Depending on the stage, a sentinel lymph node biopsy is done, as well, although controversy exists around trial evidence for this procedure. Treatment of advanced malignant melanoma is performed from a multidisciplinary approach.

4. Staging

Melanoma stages are listed below with their 5 year survival rates:

Stage 0: Melanoma in situ (Clark Level I), 99.9% survival

Stage I/II: Invasive melanoma, 85-99% survival
T1a: Less than 1.00 mm primary tumor thickness, without ulceration, and mitosis <1/mm²
T1b: Less than 1.00 mm primary tumor thickness, with ulceration or mitoses >1/mm²
T2a: 1.00-2.00 mm primary tumor thickness, without ulceration

Stage II: High risk melanoma, 40-85% survival
T2b: 1.00-2.00 mm primary tumor thickness, with ulceration
T3a: 2.00-4.00 mm primary tumor thickness, without ulceration
T3b: 2.00-4.00 mm primary tumor thickness, with ulceration
T4a: 4.00 mm or greater primary tumor thickness without ulceration
T4b: 4.00 mm or greater primary tumor thickness with ulceration

Stage III: Regional metastasis, 25-60% survival
N1: Single positive lymph node
N2: Two to three positive lymph nodes or regional skin/in-transit metastasis
N3: Four positive lymph nodes or one lymph node and regional skin/in-transit metastases

Stage IV: Distant metastasis, 9-15% survival
M1a: Distant skin metastasis, normal LDH
M1b: Lung metastasis, normal LDH
M1c: Other distant metastasis or any distant metastasis with elevated LDH

5. Prognosis

Features that affect prognosis are tumor thickness in millimeters (Breslow's depth), depth related to skin structures (Clark level), type of melanoma, presence of ulceration, presence of lymphatic/perineural invasion, presence of tumor-infiltrating lymphocytes (if present, prognosis is better), location of lesion, presence of satellite lesions, and presence of regional or distant metastasis. Certain types of melanoma have worse prognoses but this is explained by their thickness. Interestingly, less invasive melanomas even with lymph node metastases carry a better prognosis than deep melanomas without regional metastasis at time of staging. Local recurrences tend to behave similarly to a primary unless they are at the site of a wide local excision (as opposed to a staged excision or punch/shave excision) since these recurrences tend to indicate lymphatic invasion.

When melanomas have spread to the lymph nodes, one of the most important factors is the number of nodes with malignancy. Extent of malignancy within a node is also important; micrometastases in which malignancy is only microscopic have a more favorable prognosis than macrometastases. In some cases micrometastases may only be detected by special staining, and if malignancy is only detectable by a rarely employed test known as the polymerase chain reaction (PCR), the prognosis is better. Macrometastases in which malignancy is clinically apparent (in some cases cancer completely replaces a node) have a far worse prognosis, and if nodes are matted or if there is extracapsular extension, the prognosis is still worse.

When there is distant metastasis, the cancer is generally considered incurable. The five year survival rate is less than 10%. The median survival is 6 to 12 months. Treatment is palliative, focusing on life-extension and quality of life. In some cases, patients may live many months or even years with metastatic melanoma (depending on the aggressiveness of the treatment). Metastases to skin and lungs have a better prognosis. Metastases to brain, bone and liver are associated with a worse prognosis.

There is not enough definitive evidence to adequately stage, and thus give a prognosis for ocular melanoma and melanoma of soft parts, or mucosal melanoma (e.g., rectal melanoma), although these tend to metastasize more easily. Even though regression may increase survival, when a melanoma has regressed, it is impossible to know its original size and thus the original tumor is often worse than a pathology report might indicate.

6. Standard of Care Treatments

Excisional biopsies may remove the tumor, but further surgery is often necessary to reduce the risk of recurrence. Complete surgical excision with adequate surgical margins and assessment for the presence of detectable metastatic disease along with short- and long-term follow-up is standard. Often this is done by a wide local excision (WLE) with 1 to 2 cm margins. Melanoma-in-situ and lentigo malignas are treated with narrower surgical margins, usually 0.2 to 0.5 cm. Many surgeons consider 0.5 cm the standard of care for standard excision of melanoma-in-situ, but 0.2 cm margin might be acceptable for margin controlled surgery (Mohs surgery, or the double-bladed technique with margin control). The wide excision aims to reduce the rate of tumor recurrence at the site of the original lesion. This is a common pattern of treatment failure in melanoma. Considerable research has aimed to elucidate appropriate margins for excision with a general trend toward less aggressive treatment during the last decades.

Mohs surgery has been reported with cure rate as low as 77% and as high as 98% for melanoma-in-situ. CCPDMA and the “double scalpel” peripheral margin controlled surgery is equivalent to Mohs surgery in effectiveness on this “intra-epithelial” type of melanoma.

Melanomas that spread usually do so to the lymph nodes in the area of the tumor before spreading elsewhere. Attempts to improve survival by removing lymph nodes surgically (lymphadenectomy) were associated with many complications, but no overall survival benefit. Recently, the technique of sentinel lymph node biopsy has been developed to reduce the complications of lymph node surgery while allowing assessment of the involvement of nodes with tumor.

Although controversial and without prolonging survival, sentinel lymph node biopsy is often performed, especially for T1b/T2+ tumors, mucosal tumors, ocular melanoma and tumors of the limbs. A process called lymphoscintigraphy is performed in which a radioactive tracer is injected at the tumor site to localize the sentinel node(s). Further precision is provided using a blue tracer dye, and surgery is performed to biopsy the node(s). Routine hematoxylin and eosin (H&E) and immunoperoxidase staining will be adequate to rule out node involvement. Polymerase chain reaction (PCR) tests on nodes, usually performed to test for entry into clinical trials, now demonstrate that many patients with a negative sentinel lymph node actually had a small number of positive cells in their nodes. Alternatively, a fine-needle aspiration biopsy may be performed and is often used to test masses.

If a lymph node is positive, depending on the extent of lymph node spread, a radical lymph node dissection will often be performed. If the disease is completely resected, the patient will be considered for adjuvant therapy. Excisional skin biopsy is the management of choice. Here, the suspect lesion is totally removed with an adequate (but minimal, usually 1 or 2 mm) ellipse of surrounding skin and tissue. To avoid disruption of the local lymphatic drainage, the preferred surgical margin for the initial biopsy should be narrow (1 mm). The biopsy should include the epidermal, dermal, and subcutaneous layers of the skin. This enables the histopathologist to determine the thickness of the melanoma by microscopic examination. This is described by Breslow's thickness (measured in millimeters). However, for large lesions, such as suspected lentigo maligna, or for lesions in surgically difficult areas (face, toes, fingers, eyelids), a small punch biopsy in representative areas will give adequate information and will not disrupt the final staging or depth determination. In no circumstances should the initial biopsy include the final surgical margin (0.5 cm, 1.0 cm, or 2 cm), as a misdiagnosis can result in excessive scarring and morbidity from the procedure. A large initial excision will disrupt the local lymphatic drainage and can affect further lymphangiogram-directed lymph node dissection. A small punch biopsy can be used at any time where for logistical and personal reasons a patient refuses more invasive excisional biopsy. Small punch biopsies are minimally invasive and heal quickly, usually without noticeable scarring.

High-risk melanomas may require adjuvant treatment, although attitudes to this vary in different countries. In the United States, most patients in otherwise good health will begin up to a year of high-dose interferon treatment, which has severe side effects, but may improve the patient's prognosis slightly. However British Association of Dermatologist guidelines on melanoma state that interferon is not recommended as a standard adjuvant treatment for melanoma. A 2011 meta-analysis showed that interferon could lengthen the time before a melanoma comes back but increased survival by only 3% at 5 years. The unpleasant side effects also greatly decrease quality of life. In Europe, interferon is usually not used outside the scope of clinical trials.

Various chemotherapy agents also are used, including dacarbazine (also termed DTIC), immunotherapy (with interleukin-2 (IL-2) or interferon (IFN)), as well as local perfusion, are used by different centers. The overall success in metastatic melanoma is quite limited. IL-2 (Proleukin) is the first new therapy approved for the treatment of metastatic melanoma in 20 years. Studies have demonstrated that IL-2 offers the possibility of a complete and long-lasting remission in this disease, although only in a small percentage of patients. A number of new agents and novel approaches are under evaluation and show promise. Clinical trial participation should be considered the standard of care for metastatic melanoma.

For lentigo maligna treatment, standard excision is still being performed by most surgeons. Unfortunately, the recurrence rate is exceedingly high (up to 50%). This is due to the ill-defined visible surgical margin, and the facial location of the lesions (often forcing the surgeon to use a narrow surgical margin). The narrow surgical margin used, combined with the limitation of the standard “bread-loafing” technique of fixed tissue histology, result in a high “false negative” error rate, and frequent recurrences. Margin control (peripheral margins) is necessary to eliminate the false negative errors. If bread loafing is used, distances from sections should approach 0.1 mm to assure that the method approaches complete margin control.

Some melanocytic nevi, and melanoma-in-situ (lentigo maligna), have resolved with an experimental treatment: imiquimod (Aldara) topical cream, an immune enhancing agent. Some dermasurgeons are combining the 2 methods: surgically excising the cancer and then treating the area with Aldara cream postoperatively for three months.

Radiation therapy is often used after surgical resection for patients with locally or regionally advanced melanoma or for patients with unresectable distant metastases. It may reduce the rate of local recurrence but does not prolong survival. Radioimmunotherapy of metastatic melanoma is currently under investigation. Radiotherapy has a role in the palliation of metastatic melanoma.

B. Breast Cancer

Breast cancer refers to cancers originating from breast tissue, most commonly from the inner lining of milk ducts or the lobules that supply the ducts with milk. Cancers originating from ducts are known as ductal carcinomas; those originating from lobules are known as lobular carcinomas. There are many different types of breast cancer, with different stages (spread), aggressiveness, and genetic makeup; survival varies greatly depending on those factors. Computerized models are available to predict survival. With best treatment and dependent on staging, 10-year disease-free survival varies from 98% to 10%. Treatment includes surgery, drugs (hormonal therapy and chemotherapy), and radiation.

Worldwide, breast cancer comprises 10.4% of all cancer incidence among women, making it the second most common type of non-skin cancer (after lung cancer) and the fifth most common cause of cancer death. In 2004, breast cancer caused 519,000 deaths worldwide (7% of cancer deaths; almost 1% of all deaths). Breast cancer is about 100 times more common in women than in men, although males tend to have poorer outcomes due to delays in diagnosis.

Some breast cancers require the hormones estrogen and progesterone to grow, and have receptors for those hormones. After surgery those cancers are treated with drugs that interfere with those hormones, usually tamoxifen, and with drugs that shut off the production of estrogen in the ovaries or elsewhere; this may damage the ovaries and end fertility. After surgery, low-risk, hormone-sensitive breast cancers may be treated with hormone therapy and radiation alone. Breast cancers without hormone receptors, or which have spread to the lymph nodes in the armpits, or which express certain genetic characteristics, are higher-risk, and are treated more aggressively. One standard regimen, popular in the U.S., is cyclophosphamide plus doxorubicin (Adriamycin), known as CA; these drugs damage DNA in the cancer, but also in fast-growing normal cells where they cause serious side effects. Sometimes a taxane drug, such as docetaxel, is added, and the regime is then known as CAT; taxane attacks the microtubules in cancer cells. An equivalent treatment, popular in Europe, is cyclophosphamide, methotrexate, and fluorouracil (CMF). Monoclonal antibodies, such as trastuzumab (Herceptin), are used for cancer cells that have the HER2 mutation. Radiation is usually added to the surgical bed to control cancer cells that were missed by the surgery, which usually extends survival, although radiation exposure to the heart may cause damage and heart failure in the following years.

While screening techniques (which are further discussed below) are useful in determining the possibility of cancer, a further testing is necessary to confirm whether a lump detected on screening is cancer, as opposed to a benign alternative such as a simple cyst.

In a clinical setting, breast cancer is commonly diagnosed using a “triple test” of clinical breast examination (breast examination by a trained medical practitioner), mammography, and fine needle aspiration cytology. Both mammography and clinical breast exam, also used for screening, can indicate an approximate likelihood that a lump is cancer, and may also identify any other lesions. Fine Needle Aspiration and Cytology (FNAC), which may be done in a doctor's office using local anaesthetic if required, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy. Other options for biopsy include core biopsy, where a section of the breast lump is removed, and an excisional biopsy, where the entire lump is removed.

In addition vacuum-assisted breast biopsy (VAB) may help diagnose breast cancer among patients with a mammographically detected breast in women according to a systematic review. In this study, summary estimates for vacuum assisted breast biopsy in diagnosis of breast cancer were as follows sensitivity was 98.1% with 95% CI=0.972-0.987 and specificity was 100% with 95% CI=0.997-0.999. However underestimate rates of atypical ductal hyperplasia (ADH) and ductal carcinoma in situ (DCIS) were 20.9% with 95% CI=0.177-0.245 and 11.2% with 95% CI=0.098-0.128 respectively.

Breast cancer screening refers to testing otherwise-healthy women for breast cancer in an attempt to achieve an earlier diagnosis. The assumption is that early detection will improve outcomes. A number of screening test have been employed including: clinical and self breast exams, mammography, genetic screening, ultrasound, and magnetic resonance imaging.

A clinical or self breast exam involves feeling the breast for lumps or other abnormalities. Research evidence does not support the effectiveness of either type of breast exam, because by the time a lump is large enough to be found it is likely to have been growing for several years and will soon be large enough to be found without an exam. Mammographic screening for breast cancer uses x-rays to examine the breast for any uncharacteristic masses or lumps. In women at high risk, such as those with a strong family history of cancer, mammography screening is recommended at an earlier age and additional testing may include genetic screening that tests for the BRCA genes and/or magnetic resonance imaging.

Breast cancer is sometimes treated first with surgery, and then with chemotherapy, radiation, or both. Treatments are given with increasing aggressiveness according to the prognosis and risk of recurrence. Stage 1 cancers (and DCIS) have an excellent prognosis and are generally treated with lumpectomy with or without chemotherapy or radiation. Although the aggressive HER2+ cancers should also be treated with the trastuzumab (Herceptin) regime, Stage II and III cancers with a progressively poorer prognosis and greater risk of recurrence are generally treated with surgery (lumpectomy or mastectomy with or without lymph node removal), radiation (sometimes) and chemotherapy (plus trastuzumab for HER2+ cancers). Stage IV, metastatic cancer (i.e., spread to distant sites) is not curable and is managed by various combinations of all treatments from surgery, radiation, chemotherapy and targeted therapies. These treatments increase the median survival time of Stage IV breast cancer by about 6 months.

C. Dosages

In certain embodiments, the compound or compounds of the present disclosure is/are administered to a subject. In another embodiment of the disclosure, the dose range of the compound(s) will be measured by body weight, for example, about 0.5 mg/kg body weight to about 500 mg/kg body weight. Those of skill will recognize the utility of a variety of dosage range, for example, 1 mg/kg body weight to 450 mg/kg body weight, 2 mg/kg body weight to 400 mg/kg body weighty, 3 mg/kg body weight to 350 mg/kg body weighty, 4 mg/kg body weight to 300 mg/kg body weight, 5 mg/kg body weight to 250 mg/kg body weighty, 6 mg/kg body weight to 200 mg/kg body weight, 7 mg/kg body weight to 150 mg/kg body weighty, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the disclosure. Any of the above dosage ranges or dosage levels may be employed for a compound or compounds of the present disclosure.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

As is well known in the art, a specific dose level of active compounds for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

D. Formulations and Routes for Administration

Pharmaceutical compositions of the present disclosure comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present disclosure. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the disclosure, the oral composition may be prepared as a syrup or elixir. A syrup or elixir, and may comprise, for example, at least one active agent, a sweetening agent, a preservative, a flavoring agent, a dye, a preservative, or combinations thereof.

In certain particular embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

E. Combination Treatment

In the context of the present disclosure, it is contemplated that the compounds may be used in combination with each other to more effectively treat metastatic melanoma. When multiple therapeutic agents are administered, as long as the dose of the additional therapeutic agent does not exceed previously quoted toxicity levels, the effective amounts of the additional therapeutic agent may simply be defined as that amount effective to exert a therapeutic effect when administered to an animal in combination with the primary agent. This may be easily determined by monitoring the animal or patient and measuring those physical and biochemical parameters of health and disease that are indicative of the success of a given treatment. Such methods are routine in animal testing and clinical practice.

To kill or slow the growth of a cancer cell using the methods and compositions of the present disclosure, one can provide to the subject the stated combination of agents. These compositions would be provided in a combined amount effective to achieve a therapeutic benefit (inhibition of cancer cell growth, reduction in tumor size, induction of apoptosis in a cancer cell, etc.). This process may involve administering a combination at the same time. This may be achieved by administering a single composition or pharmacological formulation that includes both agents, or by administering two distinct compositions or formulations, at the same time.

Alternatively, treatment with one agent may precede or follow the additional agent treatment by intervals ranging from minutes to weeks. In embodiments where the additional agent is administered separately to the patient, one would generally ensure that a significant period of time did not expire between the time of each delivery, such that the agents would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either or both agents will be desired. Various combinations may be employed, where an inhibitor of ALDH1L1/2 or MTHFD1 or methotrexate is “A,” the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

Ccombinations with the agents/combinations include a wide variety agents. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The disclosure also encompasses the use of a combination of one or more DNA damaging agents, whether radiation-based or actual compounds, such as the use of X-rays with cisplatin or the use of cisplatin with etoposide.

In treating cancer according to the disclosure, one would contact a tumor or tumor cells with an agent according to the present disclosure along with the second agent or therapy. This may be achieved by irradiating the localized tumor site with radiation such as X-rays, UV-light, γ-rays or even microwaves. Alternatively, the tumor or tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with a compound according to the present disclosure.

Agents that directly cross-link nucleic acids, specifically DNA, are envisaged to facilitate DNA damage leading to a synergistic, antineoplastic combination with compounds of the present disclosure. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Particular agents contemplated for use in melanoma include interferon, IL-2, imiquimod, paclitaxel, vemurafenib, ipilimumab, MEK inhibitors, cardiac glycosides, BRAF inhibitors and dacarbazine.

### IV. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Methods Summary.

Melanoma specimens were obtained with informed consent from all patients according to protocols approved by the Institutional Review Boards of the University of Michigan Medical School (IRBMED approvals HUM00050754 and HUM00050085 (Quintana et al., 2012) and the University of Texas Southwestern Medical Center. Unless otherwise indicated, patient-derived xenografts were established by subcutaneously injecting 100 freshly dissociated melanoma cells into the right flanks of NOD/SCID IL2Rγ^{null} (NSG) mice in 25% high-protein Matrigel, or by injecting intravenously or intrasplenically, depending on the experiment. The subcutaneous tumors were measured every 10 days until any tumor in the mouse cohort reached 2.5 cm in its largest diameter. Mice were monitored daily for signs of distress and euthanized according to a standard body condition score or when their tumors reached 2.5 cm in largest diameter, whichever came first. Blood was collected by cardiac puncture, processed by Ficoll sedimentation, and circulating tumor cells were identified by flow cytometry. After intravenous and intrasplenic injections mice were imaged every month until the bioluminescence signal was saturated, then mice were euthanized and organs were analyzed visually and by bioluminescence imaging for the presence of micro and macrometastases. For meabolomic analyses, tumors and metastatic nodules were quickly excised and homogenized in Kontes tubes in 80% methanol on dry ice. Metabolites were extracted in two consecutive rounds of extractions, lyophilized, and resuspended in 0.03% Formic Acid. Reporters of mitochondrial mass, mitochondrial membrane potential, and ROS were used to stain equal numbers of dissociated melanoma cells and analyzed by flow cytometry. In vivo isotope tracing analysis was performed using mice fasted for 14 hours prior to injection of [U-¹³C-Glucose]. Thirty minutes after intraperitoneal injection of labeled glucose, tumors were quickly excised, and homogenized in ice cold 50% methanol. Metabolites were extracted by consecutive freeze-thaw cycles in liquid nitrogen, lyophilized, and derivatized with Trimethylsilyl (TMS). In some experiments mice received daily injections of N-acetyl-cysteine (Sigma, 200 mg/kg/day in 200 μl of PBS), or Methotrexate (Tocris, 1.25 mg/kg/day in 100 μl PBS) supplemented with Thymidine (Sigma, 3 mg/mouse/day in 100 μl PBS) and Hypoxanthine (Sigma, 750 μg/mouse/day in 100 μl of PBS) by subcutaneous injection. Tumor growth was monitored weekly until any tumor in the cohort reached 2.5 cm in its largest diameter. shRNAs were introduced into melanoma cells by lentiviral infection for 4 hours in culture. Cells were then immediately transplanted subcutaneously into the right flank of the mice. The resulting tumors were dissociated, shRNA-infected cells were isolated by flow cytometry (based on GFP or dsRed expression), and subcutaneously injected into right flank of mice for experiments (100 cells/mouse).

Obtaining Melanomas and Enzymatic Dissociation.

Melanoma specimens were obtained with informed consent from all patients according to protocols approved by the Institutional Review Boards of the University of Michigan Medical School (IRBMED approvals HUM00050754 and HUM00050085; see ref (Quintana et al., 2012)) and the University of Texas Southwestern Medical Center. Tumors were dissociated in Sterile Closed System Tissue Grinders (SKS Science) in enzymatic digestion medium containing 200 U/ml collagenase IV (Worthington) for 20 minutes at 37° C. DNase (50-100 U/ml) was added to reduce clumping of cells during digestion. Cells were filtered with a 40 μm cell strainer to obtain a single-cell suspensions.

Cell Labeling and Sorting.

All antibody labeling of cells was performed for 20 minutes on ice, followed by washing and centrifugation. Cells were stained with directly conjugated antibodies to mouse CD45 (30-F11-APC, eBiosciences), mouse CD31 (390-APC, Biolegend), Ter119 (TER-119-APC, eBiosciences) and human HLA-A,B,C (G46-2.6-FITC, BD Biosciences) to select live human melanoma cells and to exclude contaminating mouse endothelial and haematopoietic cells. Prior to flow cytometric analysis, cells were re-suspended in staining medium (L15 medium containing bovine serum albumin (1 mg/ml), 1% penicillin/streptomycin, and 10 mM Hepes (pH 7.4)) containing 4′,6-diamidino-2-phenylindole (DAPI; 5 μg/ml; Sigma) to eliminate dead cells from sorts and analyses. Sorts and analyses were performed using a FACSAria flow cytometer (Becton Dickinson). After sorting, an aliquot of sorted melanoma cells was always reanalysed to check for purity, which was usually greater than 95%. For analysis of circulating melanoma cells, blood was collected from each mouse by cardiac puncture with a syringe pretreated with citrate-dextrose solution (Sigma). Red blood cells were precipitated by Ficoll sedimentation according to the manufacturer's instructions (Ficoll Paque Plus, GE Healthcare). Remaining cells were washed with Hanks' balanced salt solution (Invitrogen) before antibody staining and flow cytometric analysis. For limiting dilution analysis, cells for each mouse were sorted into individual wells of 96-well V-bottomed plates containing staining medium and loaded into syringes directly from the well (one well into one syringe into one mouse).

Transplantation of Melanoma Cells.

After sorting, cells were counted and resuspended in staining medium with 25% high-protein Matrigel (product 354248; BD Biosciences). Subcutaneous injections were performed into the right flank of NOD.CB17-Prkdcscid Il2rgtmlWj1/SzJ (NOD/SCID IL2Rγnull or NSG) mice (Jackson Laboratory) in a final volume of 50 pl. Each mouse was transplanted with 100 melanoma cells unless otherwise specified. Tumor formation was evaluated regularly by palpation of the injection site, and tumor diameters were measured every 10 days with calipers. Mice were monitored until subcutaneous tumor diameter reached 2 to 2.5 cm then mice were sacrificed and tumors were removed by surgery. Organs were analyzed visually and by bioluminescence imaging (see details below) for presence of macrometastases and micrometastases. These experiments were performed according to protocols approved by the Institutional Animal Care and Use Committee at the University of Texas Southwestern Medical Center (protocol 2011-0118). Intravenous injections were done by injecting cells into the tail vein of NSG mice in 100 μl of staining medium. For intrasplenic injections the mice were anesthetized with isoflourane, then the left flank was shaved and disinfected with an ethanol wipe and iodine swab. An incision was made into the intraperitoneal cavity. The spleen was exposed with forceps and cells were injected slowly in a 40 μl volume of staining medium. The peritoneum was then sutured and skin was closed with clips. Mice were injected with buprenex before surgery and then again 12 hours after surgery.

Lentiviral Transduction of Human Melanoma Cells.

A bicistronic lentiviral construct carrying dsRed2 and luciferase (dsRed2-P2A-Luc) was generated (for bioluminescence imaging) and cloned into the FUW lentivrial expression construct. The primers that were used for generating this construct were: dsRed2 forward, 5′-CGACTCTAGAGGATCCatggatagcactgagaacgtc-3′ (capital letters indicate homology to FUW backbone); dsRed2 reverse, 5′-TCCACGTCTCCAGCCTGCTTCAGCAGGCTGAAGTTAGTAGCTCCGCTTCCctggaaca ggtggtggc-3′ (capital letters indicate P2A sequences); luciferase forward, 5′-GCCTGCTGAAGCAGGCTGGAGACGTGGAGGAGAACCCTGGACCTGGATCCatggaa gacgccaaaaacataaag-3′ (capital letters indicate P2A sequences) and luciferase reverse, 5′-GCTTGATATCGAATTCttacacggcgatctttccgc-3′ (capital letters indicate homology to FUW backbone). All constructs were generated using the In-Fusion HD cloning system (Clontech) and sequence verified.

For virus production, 0.9 μg of the appropriate plasmid together with 1 μg of helper plasmids (0.4 μg pMD2G and 0.6 μg of psPAX2) were transfected to 293T cells using polyjet (Signagen) according to the manufacturers instructions. The resulting replication incompetent viral supernatants were collected 48 hours after transfection and filtered through a 0.45 μm filter. 300,000 freshly dissociated melanoma cells were infected with viral supernatants supplemented with 10 μg/ml poybrene (Sigma) for 4 hours. Cells were then washed twice with staining medium, and about 25,000 cells (a mixture of infected and noninfected cells) were suspended in staining medium with 25% high-protein Matrigel (product 354248; BD Biosciences) then injected subcutaneously into NSG mice. After growing to 1 to 2 cm in diameter, tumors were excised and dissociated into single-cell suspensions, and luciferase-dsRed⁺ or GFP⁺ cells were collected by flow cytometry for injection into secondary recipients. Metastasis was monitored by bioluminescence imaging in secondary recipients.

Bioluminescence Imaging.

Mice were injected with 100 luciferase-dsRed⁺ cells on the right flank and monitored until tumor diameters approached 2.5 cm, at which point they were imaged along with an uninjected control mouse using an IVIS Imaging System 200 Series (Caliper Life Sciences) with Living Image software. Mice were injected intraperitoneally with 100 μl of PBS containing D-luciferin monopotassium salt (40 μg/ml) (Biosynth) 5 minutes before imaging, followed by general anesthesia 2 minutes before imaging. After imaging of the whole mouse, the mice were euthanized and individual organs were surgically removed and quickly imaged. The exposure time of images ranged from 10 to 60 seconds depending on signal intensity. The bioluminescence signal was quantified with “region of interest” measurement tools in Living Image (Perkin Elmer) software. After imaging, tumors and organs were fixed in 10% neutral-buffered formalin for histopathology.

LC-MS Metabolomic Analysis.

Mice were euthanized by cervical dislocation.

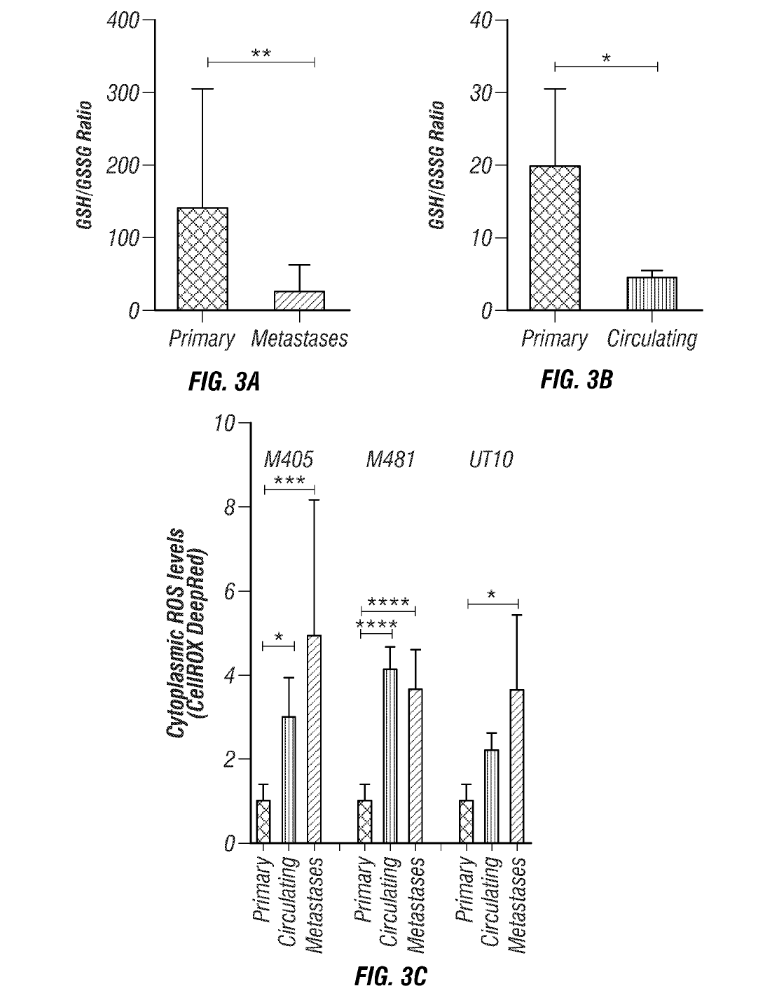

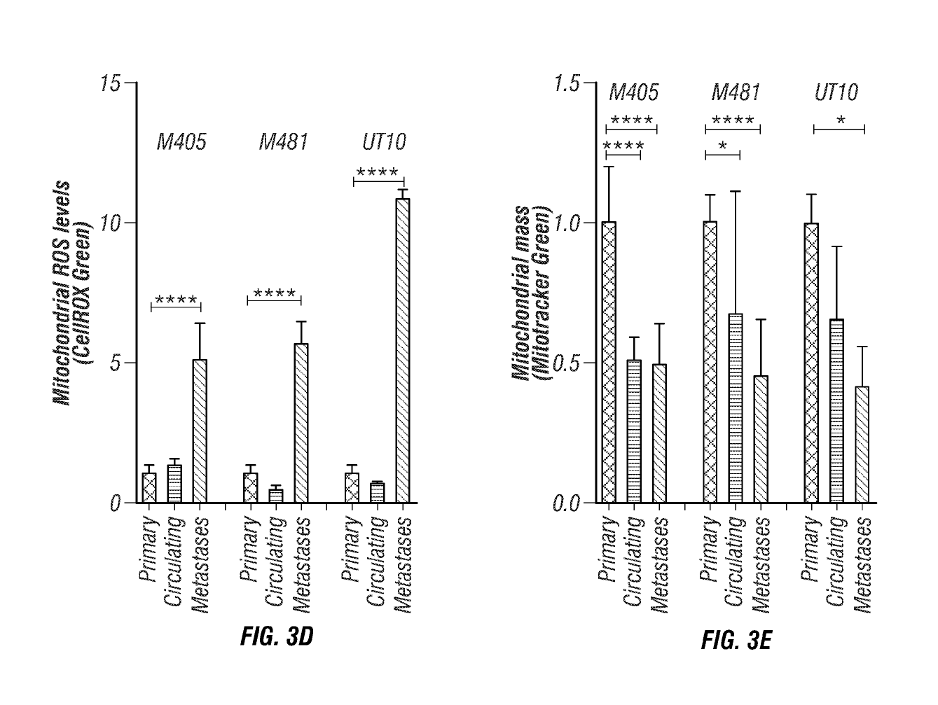

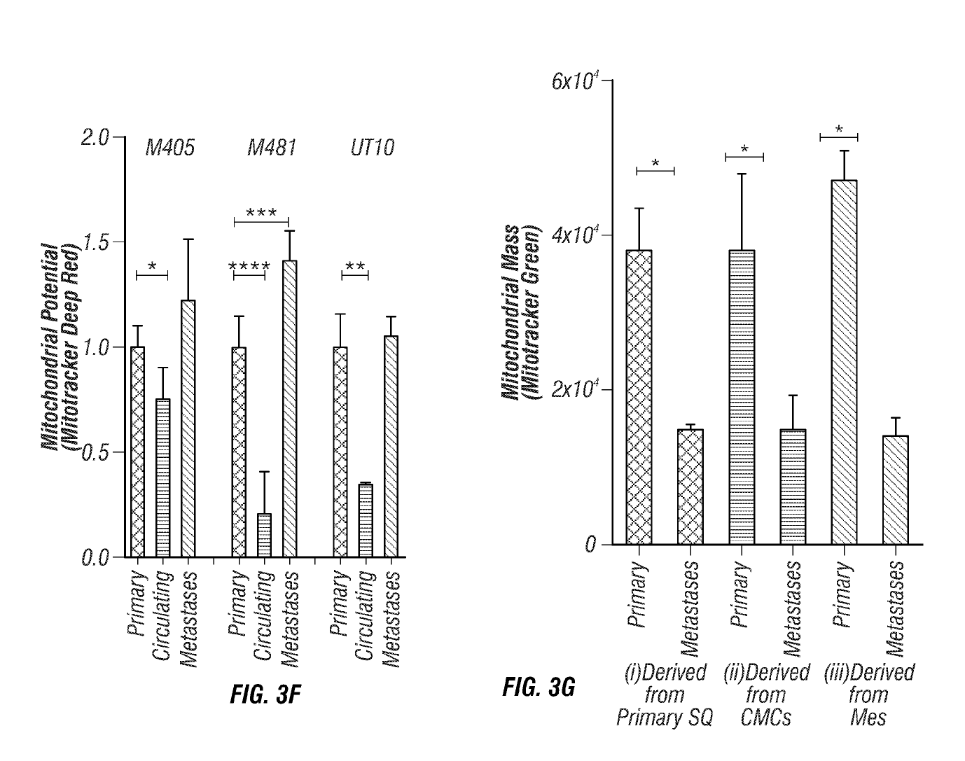

Primary tumors and metastatic nodules were dissected, immediately homogenized in 80% methanol chilled with dry ice (Honeywell), vortexed vigorously, and metabolites were extracted overnight at −80° C. The following day, samples were centrifuged at 13,000×g for 15 minutes at 4° C., the supernatant was collected, and metabolites from the pellet were re-extracted with 80% methanol at −80° C. for 4 hours. After centrifugation, both supernatants were pooled and lyophilized using a SpeedVac (Thermo). Dried metabolites were reconstituted in 0.03% formic acid in water, vortexed and centrifuged, then the supernatant was analysed using liquid chromatography-tandem mass spectrometry (LC-MS/MS). A Nexera Ultra High Performance Liquid Chromatograph (UHPLC) system (Shimadzu) was used for LC, with a Polar-RP HPLC column (150×2 mm, 4 μm, 80 Å, Phenomenex) and the following gradient: 0-3 min 100% mobile phase A; 3-15 min 100%-0% A; 15-17 min 0% A; 17-18 min 0%-100% A; 18-23 min 100% A. Mobile Phase A was 0.03% formic acid in water. Mobile Phase B was 0.03% formic acid in acetonitrile. The flow rate was 0.5 ml/min and the column temperature 35° C. A triple quadrupole mass spectrometer (AB Sciex QTRAP 5500) was used for metabolite detection as previously described⁶⁴. Chromatogram peak areas were integrated using Multiquant (AB Sciex). For GSH and GSSG measurements, metabolite extractions were repeated with 0.1% Formic acid in 80% methanol, to prevent spontaneous GSH oxidation, and data were consistent with those shown in FIG. 3A.

Flow Cytometric Analysis of Mitochondrial Mass, Mitochondrial Membrane Potential and ROS.

Melanomas were generally dissociated enzymatically as described above. Equal numbers of dissociated cells (500,000-2,000,000) from subcutaneous tumors, Ficoll-depleted blood, or metastatic nodules were loaded with dyes to assess mitochondrial mass, mitochondrial membrane potential, and ROS levels. The dyes that were used to assess these parameters were all obtained from Life Technologies. The inventors stained the dissociated cells for 20-45 minutes at 37° C. with 5 μM Mitotracker Green, Mitotracker DeepRed, CellROX Green, or CellROX DeepRed in HBSS-free (Ca⁺ and Mg²⁺ free) to assess mitochondrial mass, mitochondrial membrane potential, mitochondrial and cytoplasmic ROS, respectively. For each indicator, staining intensity per cell was assessed by flow cytometry in live human melanoma cells (positive for human HLA and dsRed and negative for DAPI and mouse CD45/CD31/Ter119).

In Vivo Treatment of Xenografts with Drugs.

All animal experiments were performed according to protocols approved by the Institutional Animal Care and Use Committee at the University of Texas Southwestern Medical Center (protocol 2011-0118). Unless otherwise stated, 100 freshly dissociated melanoma cells were injected subcutaneously into the right flanks of NSG mice. When tumors became palpable, in some experiments mice were injected subcutaneously with N-acetyl-cysteine (NAC) (Sigma, 200 mg/kg/day in 200 μl PBS, pH 7.4) or PBS as a control. Mice were injected with their last NAC dose 10 minutes before being sacrificed for end point analysis. In other experiments methotrexate (Tocris, 1.25 mg/kg/day in 100 μl PBS) was injected intraperitoneally 5 days per week. Mice that received methotrexate were simultaneously administered Thymidine (Sigma, 3 mg/mouse/day in 100 μl PBS) and Hypoxanthine (Sigma, 750 μg/mouse/day in 100 μl PBS) to prevent suppression of nucleotide biosynthesis. Tumor growth was monitored weekly with a caliper. Experiments were terminated when any tumor in the cohort reached 2.5 cm in size. At the end of experiments, blood was collected by cardiac puncture. Organs were analyzed for micrometastases and macrometastases by bioluminescence imaging and visual inspection.

NADPH/NADP⁺ Measurement.

Primary subcutaneous tumors or metastatic nodules were surgically excised as quickly as possible after euthanizing the mice then melanoma cells were mechanically dissociated and NADPH and NADP+ were measured using NADPH/NADP Glo-Assay (Promega) following the manufactures instructions. Luminescence was measured using a using a FLUOstar Omega plate reader (BMG Labtech). Values were normalized to protein concentration, measured using a bicinchoninic acid (BCA) protein assay (Thermo).

Isotope tracing with [U-¹³C]glucose. Mice were injected intraperitoneally with 2 g/kg body mass of [U-¹³C]Glucose (Cambridge Isotopes) and were analyzed 30-45 minutes later. Mice were fasted for 14 hours prior to the injection. Primary subcutaneous tumor tissue and metastatic nodules were surgically excised and homogenized in ice cold 50% methanol. Metabolites were extracted with three freeze-thaw cycles in liquid nitrogen. Supernatant was collected after a 15 minute centrifugation at 13000×g at 4° C. and lyophilized. Metabolites were derivatized with Trimethylsilyl (TMS) at 42° C. for 30 minutes. (Debnath & Brugge, 2005) C enrichment analysis was performed by GC-MS as previously described (Mullen et al., 2014).

Western Blot Analysis.

Tissue lysates were prepared in Kontes tubes with disposable pestles using RIPA Buffer (Cell Signaling Technology) supplemented with phenylmethylsulphonyl fluoride (Sigma), and protease and phosphatase inhibitor cocktails (Roche). The BCA protein assay (Thermo) was used to quantify protein concentrations. Equal amounts of protein (15-30 μg) were separated on 4-20% Tris Glycine SDS gels (BioRad) and transferred to polyvinylidene difluoride membranes (BioRad). Membranes were blocked for 30 minutes at room temperature with 5% milk in TBS supplemented with 0.1% Tween20 (TBST) then incubated with primary antibodies overnight at 4° C. After incubating with horseradish peroxidase conjugated secondary antibodies (Cell Signaling Technology), membranes were developed using SuperSignal West Pico or Femto chemiluminescence reagents (Thermo). Blots were stripped with 1% SDS, 25 mM glycine (pH 2) before re-probing. The following primary antibodies were used for western blot analyses: ALDH1L1 (LifeSpan Bio; LS-C172406), ALDH1L2 (LifeSpan Bio; LS-C178510), DHFR (LifeSpan Bio; LS-C138829), MTHFR (LifeSpan Bio; LS-C157974), SHMT1 (Cell Signaling; 12612S), SHMT2 (Cell Signaling; 12762S), MTHFD1 (ProteinTech; 10794-1-AP), MTHFD2 (ProtenTech; 12270-1-AP), Actin (Abcam, ab8227).

Statistical Methods.

The data in most figure panels reflect multiple independent experiments performed on different days using melanomas derived from multiple patients. Variation is always indicated using standard deviation. For analysis of statistical significance, the inventors first tested whether there was homogeneity of variation across treatments (as required for ANOVA) using Levene's test, or when only two conditions were compared, using the F-test. In cases where the variation significantly differed among treatments, the data were log 2-transformed. If the data contained zero values, 1/2 of the smallest non-zero value was added to all measurements before log 2 transformation. If the data contained negative values, all measurements were log-modulus transformed (L(x)=sign(x)*log(|x|+1)). In the rare cases when the transformed data continued to exhibit variation that significantly differed among treatments, the inventors used a non-parametric Kruskal-Wallis test or a non-parametric Mann-Whitney test to assess the significance of differences among populations and treatments. Usually, variation did not significantly differ among treatments. Under those circumstances, two-tailed Student's t-tests were used to test the significance of differences between two treatments. When more than two treatments were compared, a one-way ANOVA followed by Dunnett's multiple comparisons tests were performed. A two-way ANOVA followed by Dunnett's multiple comparisons tests were used in cases where more than two groups were compared with repeated measures. Hierarchical clustering was analyzed using Metaboanalyst⁶⁵.

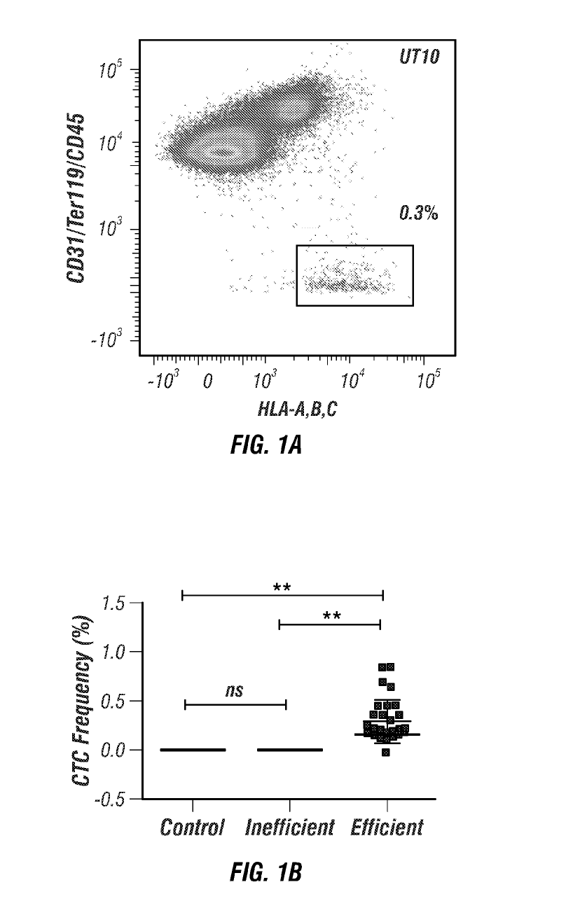

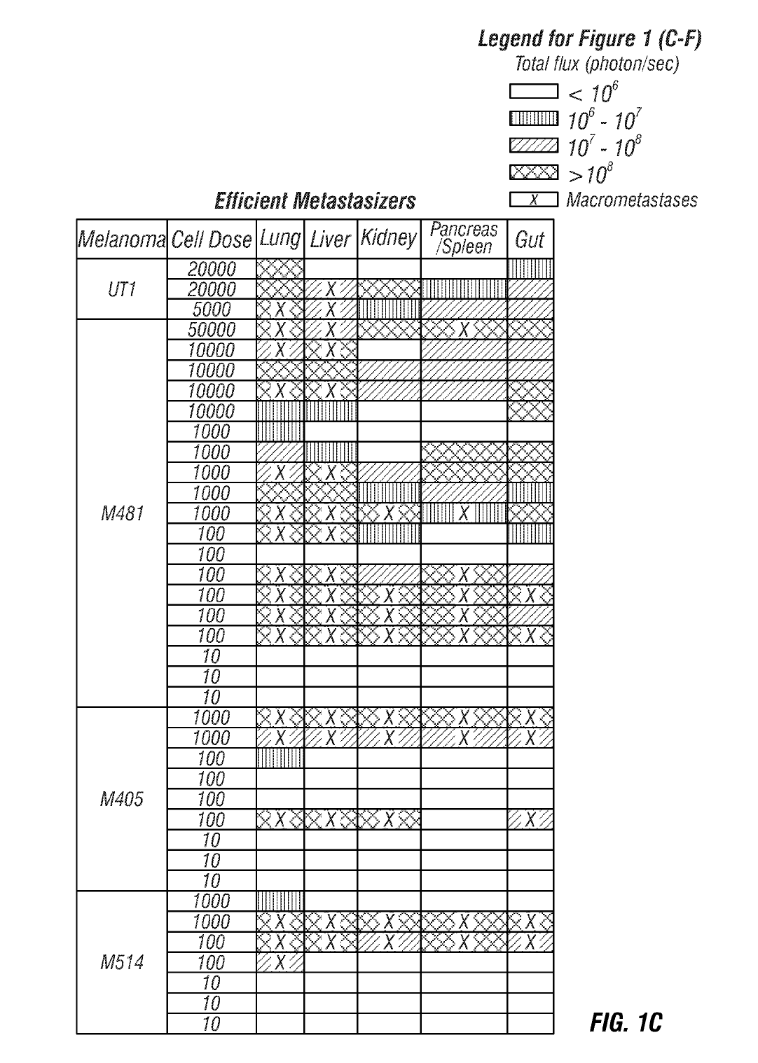

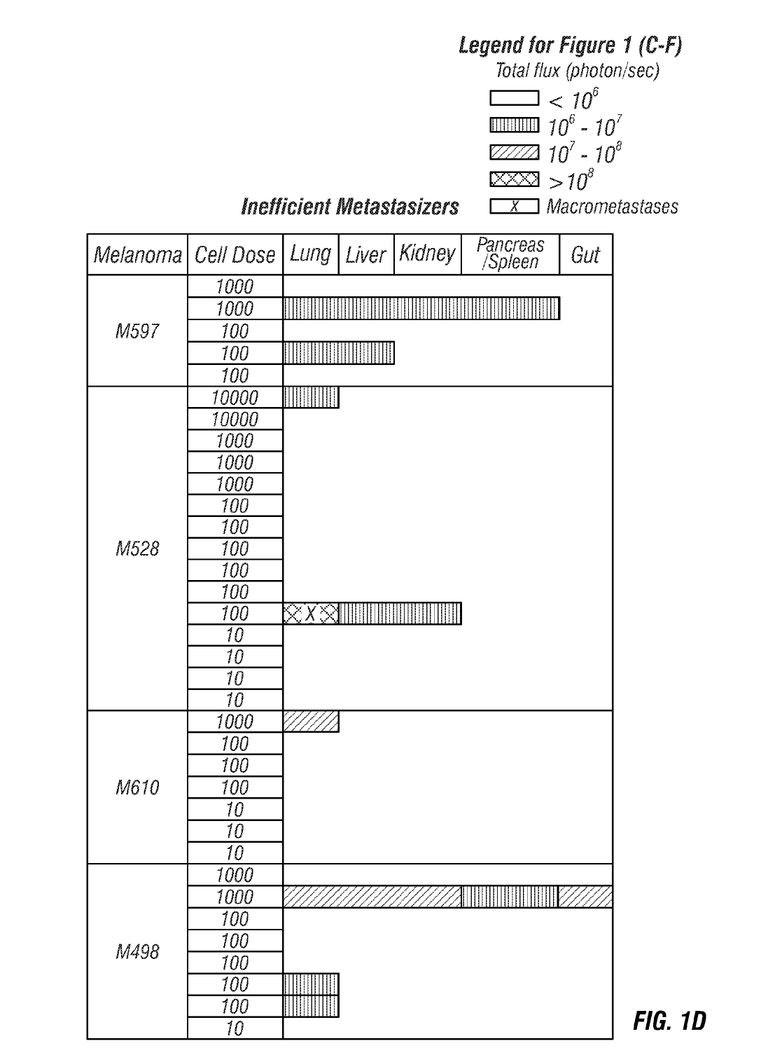

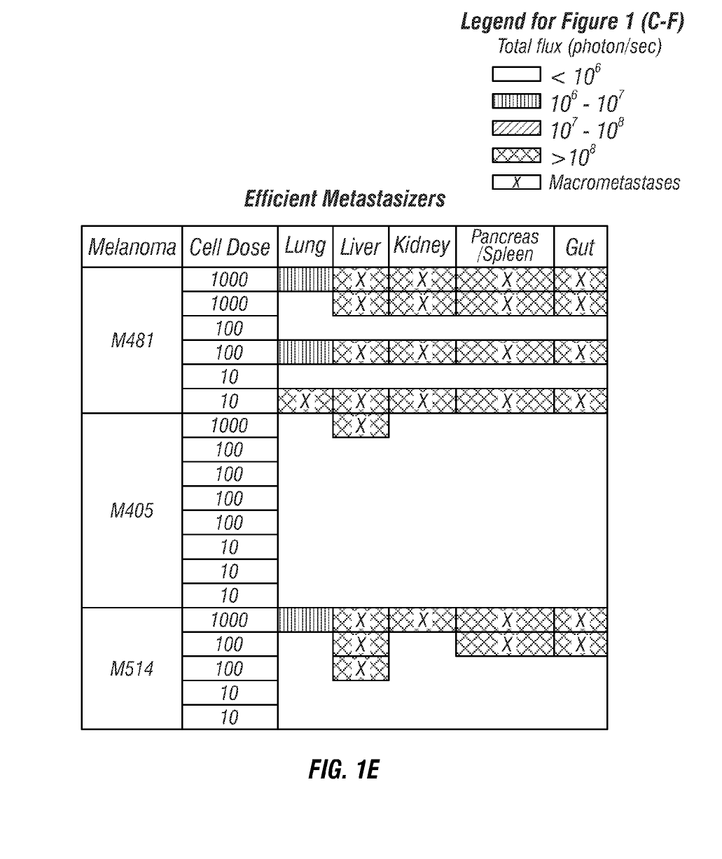

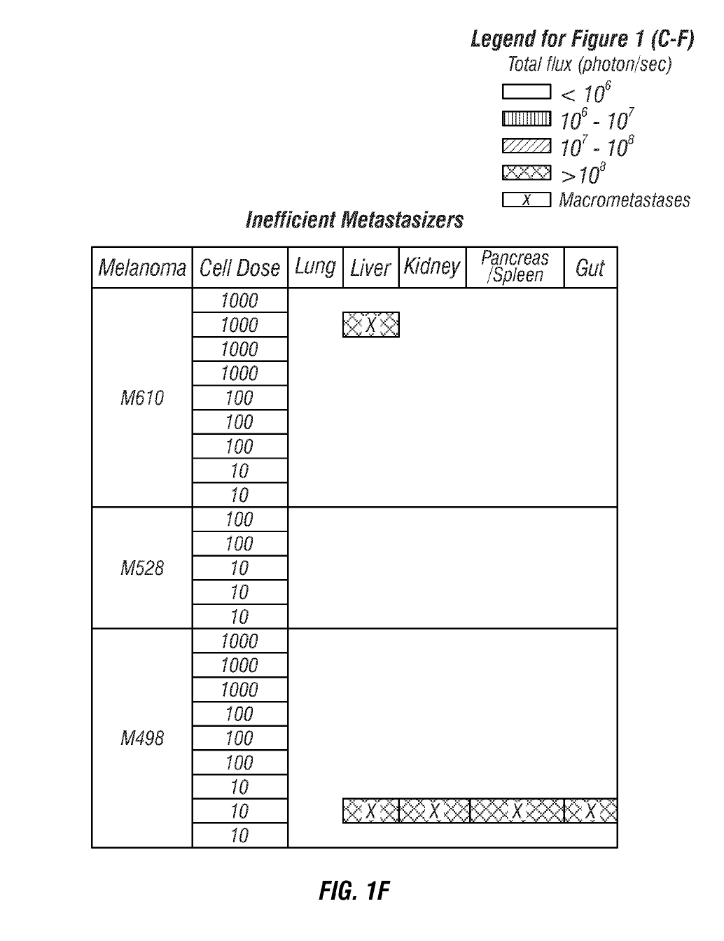

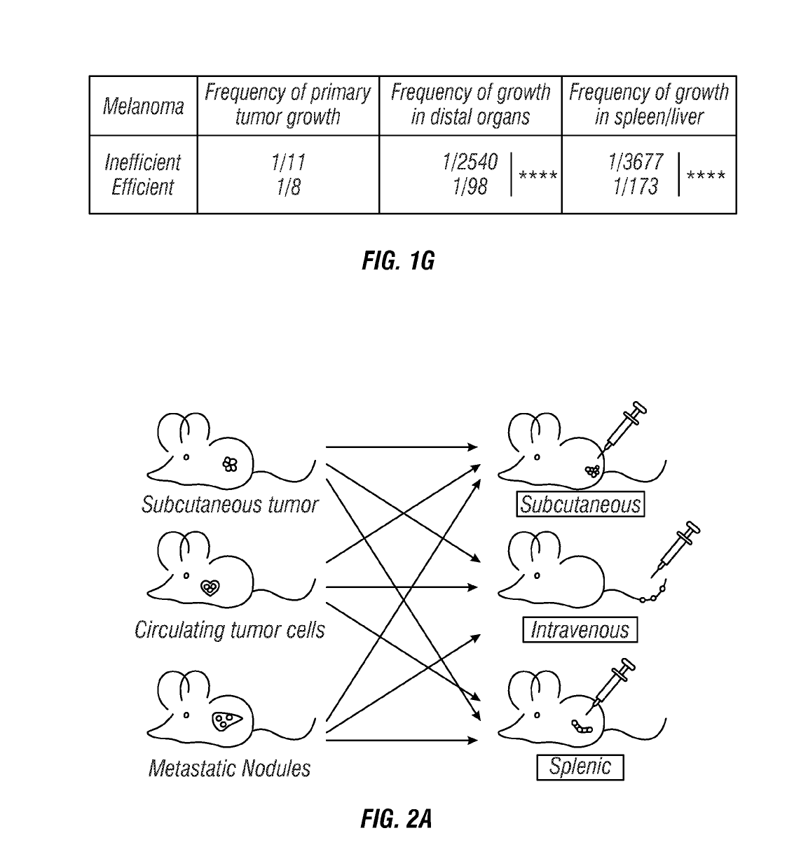

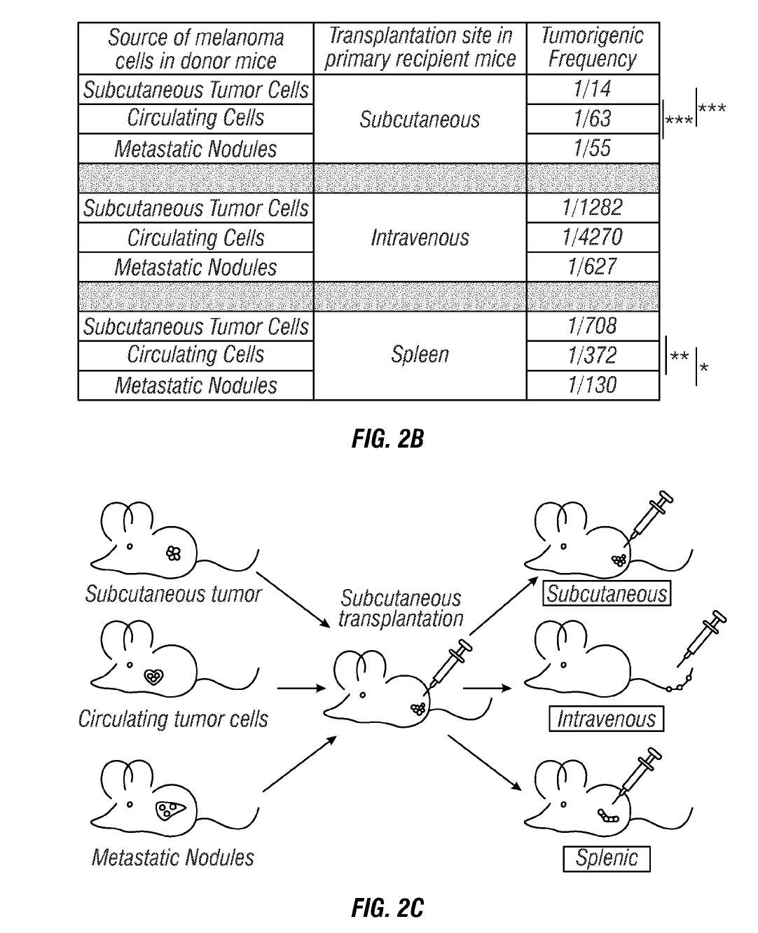

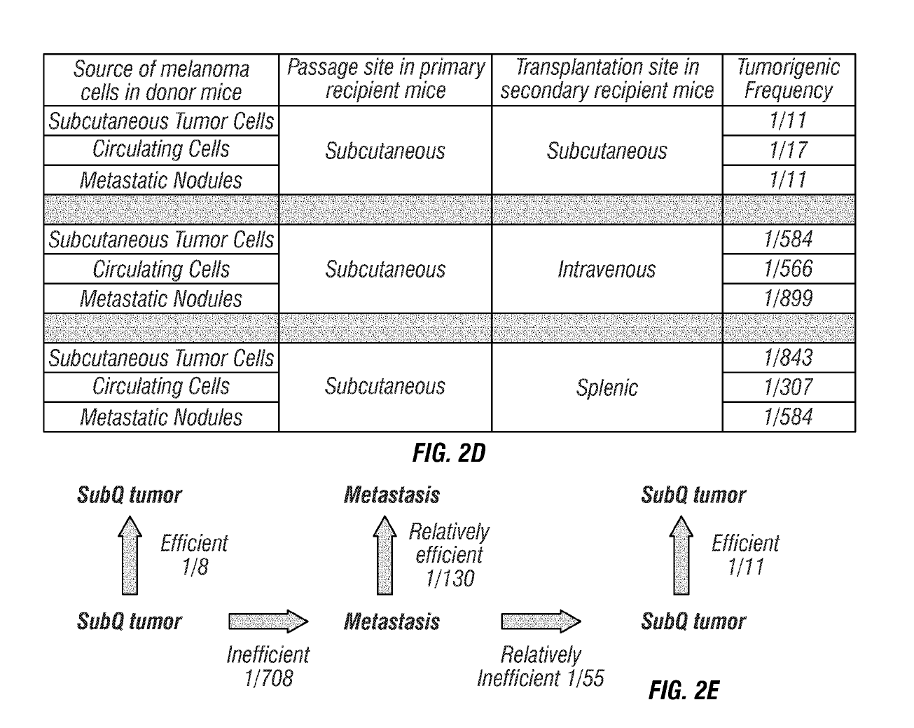

Mouse cages were randomized between treatments in all in vivo experiments (mice within the same cage had to be part of the same treatment). No blinding was used in any experiment. In all xenograft assays the inventors injected 4-8 week old mice, 5 mice per treatment. For long-term assays, they injected 10 mice per treatment to account for non-melanoma related deaths (NSG mice are susceptible to death from opportunistic infections). When mice died before the end of experiments due to opportunistic infections the data from those mice were excluded. There were only two experiments in which this occurred. In FIGS. 1C-D, 0-4 mice per melanoma line were found dead due to an opportunistic bacterial infection prior to termination of the experiment and were excluded from the reported results. In FIG. 2B, 0-3 mice per melanoma line were found dead due to opportunistic infections, before the first imaging time point after transplantation. These mice were excluded from the reported results.

### Example 2—Results

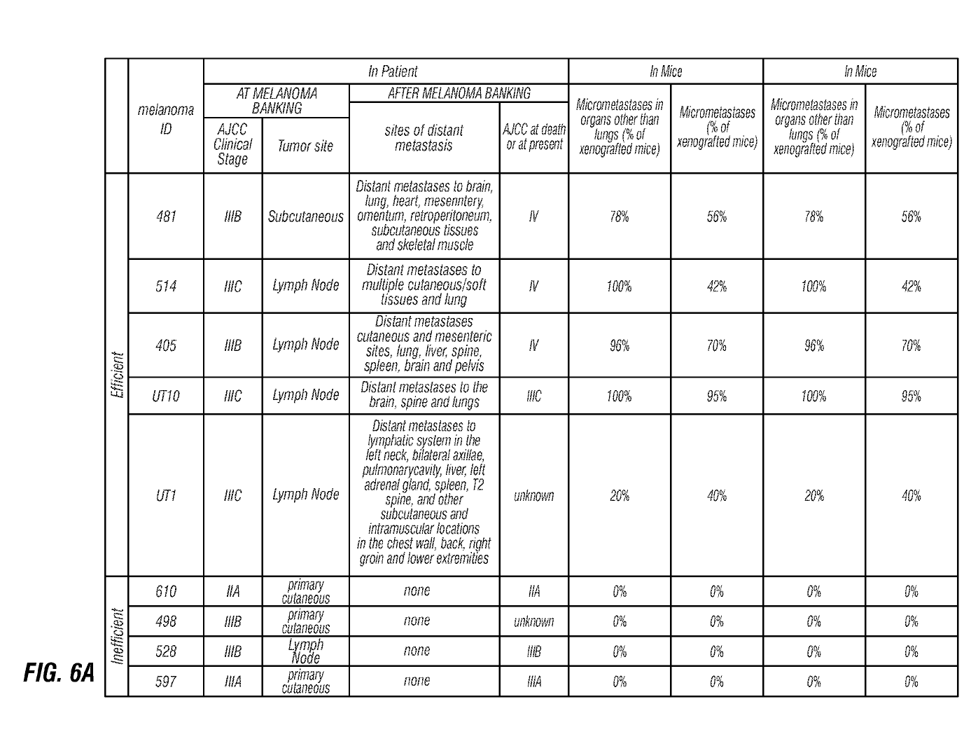

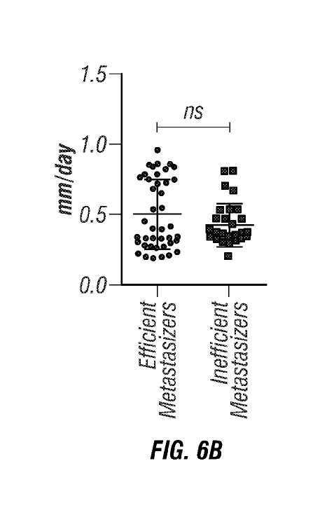

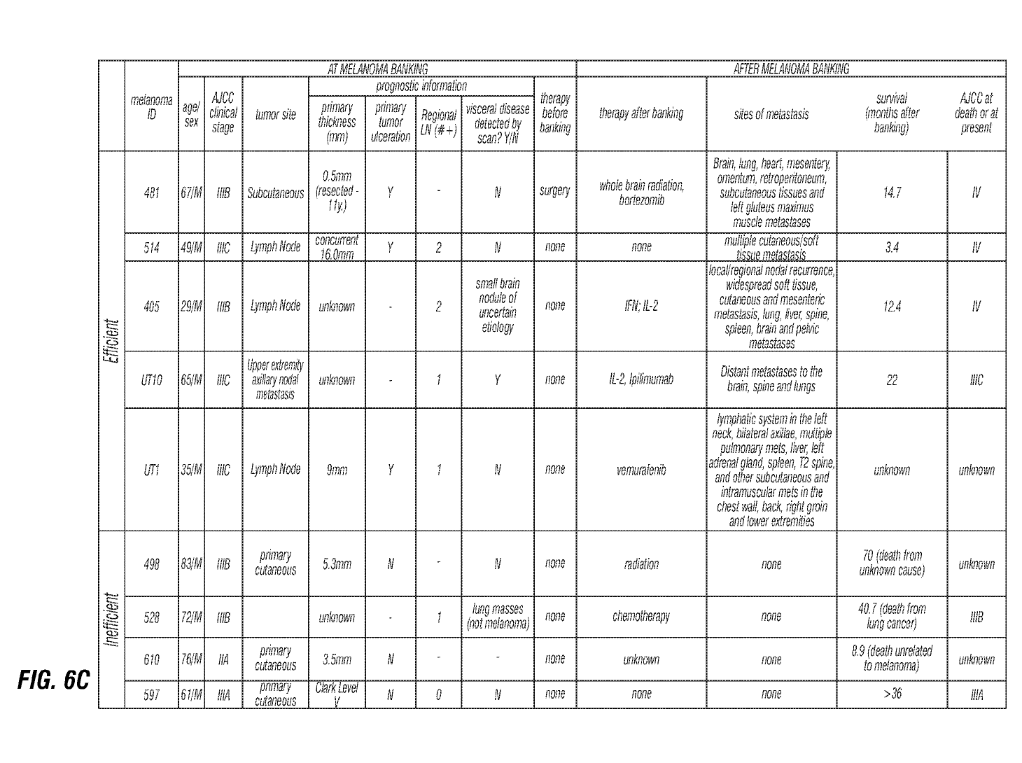

Blood and visceral organs are hostile to metastasis. The inventors obtained efficiently metastasizing melanomas from five patients (UT1, UT10, M481, M405, and M514) and inefficiently metastasizing melanomas from four patients (M597, M528, M610, and M498). Most of the efficiently metastasizing melanomas were regional lymph node metastases from patients with stage IIIb/c disease (FIG. 6A). Each of these melanomas formed distant metastases in the patients and reproducibly formed distant macrometastases in NOD.Cg-Prkdc^{scid} Il2rg^{tm1Wjl}/SzJ (NSG) mice after subcutaneous injection (FIG. 6A). The inefficiently metastasizing melanomas were obtained as primary cutaneous tumors or regional lymph node metastases from patients with stage IIa, IIIa, and IIIb disease (FIG. 6A). These melanomas did not form distant metastases in patients or macrometastases in NSG mice in these experiments (FIG. 6A).

Despite exhibiting intrinsic differences in their propensity to metastasize in humans and NSG mice, the efficient and inefficient metastasizers did not significantly differ with regard to the frequency of cells that formed tumors upon subcutaneous injection or the rate at which these tumors grew. An average of 1 in 8 cells from efficiently metastasizing melanomas and 1 in 11 cells from inefficiently metastasizers formed tumors upon subcutaneous injection in NSG mice (FIG. 1G). Efficiently metastasizing melanomas formed subcutaneous tumors that grew at 0.51±0.25 mm/day while inefficiently metastasizing melanomas formed subcutaneous tumors that grew at 0.43±0.15 mm/day (FIG. 6B). Cells with tumor-forming capacity are thus common in both efficiently and inefficiently metastasizing melanomas.

To better understand the mechanisms underlying the difference in metastatic potential, the inventors subcutaneously injected NSG mice with 100 melanoma cells derived from each of six different patients. They allowed the resulting tumors to grow to 2.5 cm in diameter and then assessed the frequency of circulating melanoma cells (CMCs) in the blood of these mice by flow cytometry. The inventors often detected human melanoma cells in the blood of mice with efficiently metastasizing melanomas but not in mice with inefficiently metastasizing melanomas (FIGS. 1A-B). This is consistent with the inventors' prior work (Quintana et al., 2012) in showing that one difference between efficient and inefficient metastasizers is the ability to intravasate or survive in circulation.

If the main difference between efficient and inefficient metastasizers is the ability to intravasate into circulation, the inventors reasoned that intravenous injection would lead both types of melanomas to efficiently metastasize. The inventors therefore injected 10, 100, 1000, or 10,000 cells from efficiently metastasizing or inefficiently metastasizing (four patients each) melanomas into NSG mice. The melanoma cells were marked by luciferase expression, allowing us to confirm micro and macrometastases by bioluminescence imaging. Efficiently metastasizing melanomas from all four patients formed macrometastases in multiple visceral organs (FIG. 1C). Limiting dilution analysis thus indicated that at least 1 in 98 cells from efficient metastasizers formed tumors upon intravenous injection. In contrast, inefficiently metastasizing melanomas rarely formed tumors after intravenous injection even when the inventors injected 1,000 or 10,000 cells (FIG. 1D). Limiting dilution analysis indicated that only 1 in 2,540 cells from inefficiently metastasizers formed tumors upon intravenous injection. Therefore the ability to intravasate into circulation is not the only factor that limits distant metastasis because efficient metastasizers much more readily (p<0.0001) formed tumors than inefficient metastasizers even after intravenous injection. This suggests that the ability to survive in circulation, or the ability to extravasate or proliferate in visceral organs, also limits distant metastasis.

These data also demonstrate that even efficiently metastasizing melanomas more readily formed tumors after subcutaneous injection (1 in 8 cells) as compared to intravenous injection (1 in 98 cells, FIG. 1G). This suggests that the blood was a more hostile environment for melanoma cells as compared to the subcutaneous environment in these experiments.

If the main difference between efficient and inefficient metastasizers is the ability to survive in circulation, the invetors reasoned that direct injection into a visceral organ would lead both types of melanomas to efficiently form visceral tumors. To test this, the inventors injected efficiently metastasizing (M481, M405, and M514) or inefficiently metastasizing (M610, M528, and M498) melanomas from three patients each directly into the spleens of NSG mice. Efficiently metastasizing melanomas from two of three patients formed macrometastases in multiple visceral organs in most mice (FIG. 1E). Limiting dilution analysis indicated that at least 1 in 173 cells from efficient metastasizers formed tumors upon intrasplenic injection (FIG. 1G). In contrast, inefficiently metastasizers rarely formed tumors after intrasplenic injection even when the inventors injected 1,000 cells (FIG. 1F). Limiting dilution analysis indicated that only 1 in 3677 cells from inefficient metastasizers formed tumors upon splenic injection (FIG. 1G). The ability to survive in circulation is therefore not the only factor that distinguishes efficient from inefficient metastasizers. Since even efficiently metastasizing melanomas more readily formed tumors after subcutaneous injection (1 in 8 cells) as compared to intrasplenic injection (1 in 173 cells, FIG. 1G) visceral organs were also a relatively hostile environment for melanoma cells.

Melanoma Cells Undergo Reversible Changes in Tropism During Metastasis.

To test whether melanoma cells undergo changes during metastasis that influence their ability to form tumors at distant sites, the inventors obtained efficiently metastasizing melanoma cells from 12 donor mice that had been grafted with melanomas derived from 3 patients (M481, M405 and UT10). The inventors compared the capacity of melanoma cells from subcutaneous tumors versus the blood versus metastatic liver nodules (2-5 mm in diameter) in the same donor mice to form tumors upon subcutaneous, intravenous, or intrasplenic injection in recipient mice (FIG. 2A). Subcutaneous melanoma cells were significantly (p<0.001) better at forming subcutaneous tumors (1 in 14 cells formed tumors) as compared to melanoma cells from the blood (1 in 63 cells) or metastatic nodules (1 in 55 cells; FIG. 2A). In contrast, melanoma cells from metastatic nodules were significantly (p<0.05) better at forming tumors upon intrasplenic injection (1 in 130 cells) as compared to melanoma cells from the blood (1 in 372 cells) or subcutaneous tumors (1 in 708 cells; FIG. 2B). Subcutaneous melanoma cells are therefore more effective than circulating or metastatic cells at forming subcutaneous tumors while visceral metastatic cells are more effective than subcutaneous cells at forming visceral tumors. This suggests melanoma cells undergo adaptive changes as they metastasize.

To test whether the underlying mechanisms reflected irreversible (i.e., genetic) or reversible (i.e., epigenetic or metabolic) changes, the inventors obtained melanoma cells from subcutaneous tumors, the blood, or metastatic liver nodules of the same donor mice (2 mice transplanted with melanomas derived from patient M481). The inventors transplanted these cells subcutaneously into primary recipient mice and allowed them to form a tumor (see FIG. 2C for experimental design). Then the inventors retransplanted dissociated melanoma cells subcutaneously, intravenously, or intrasplenically into secondary recipient mice (85 total) to test whether small numbers of melanoma cells from any site would reacquire subcutaneous properties after being passaged subcutaneously. Melanoma cells derived from all sites formed subcutaneous tumors with indistinguishably high efficiency in secondary recipient mice after being passaged subcutaneously in primary recipient mice (FIG. 2D). Melanoma cells derived from all sites also formed metastatic tumors with indistinguishably low efficiency in secondary recipient mice after being passaged subcutaneously in primary recipient mice (FIG. 2D). The changes in tumorigenic tropism that melanoma cells undergo as they metastasize are thus reversible.

Oxidative Stress Limits Distant Metastasis.

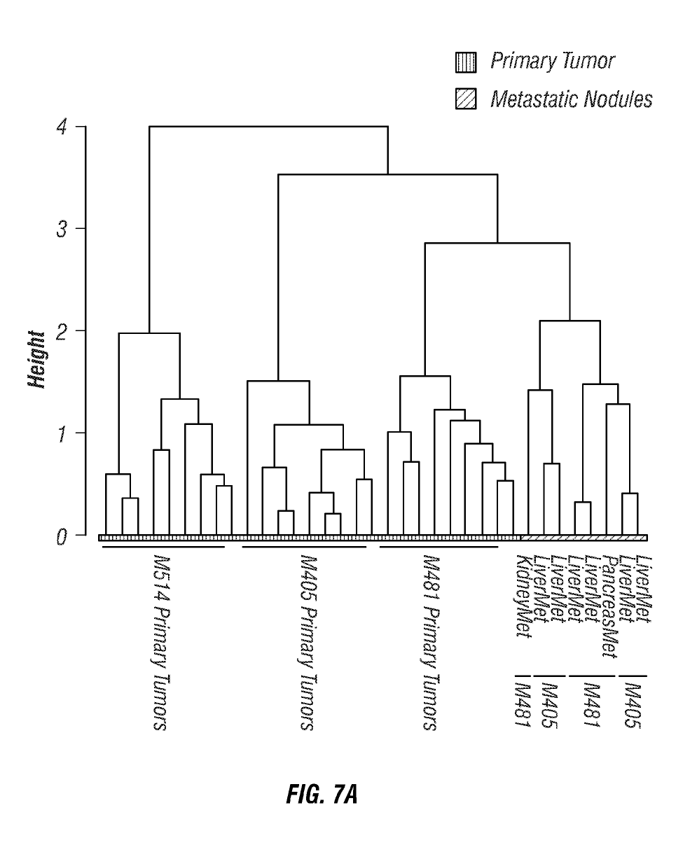

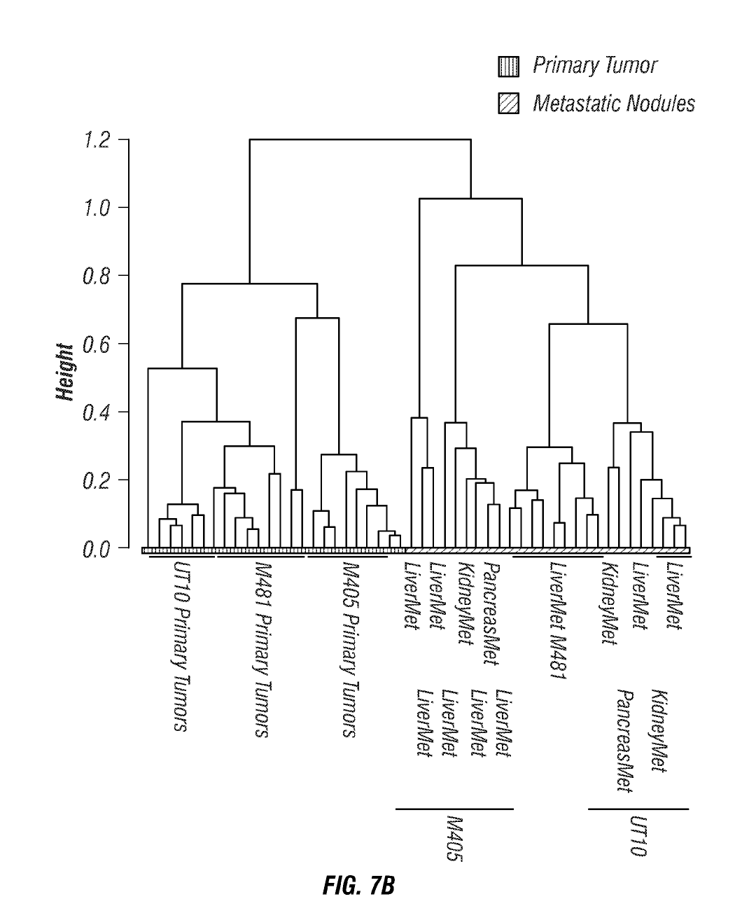

To test whether melanoma cells underwent reversible metabolic changes during metastasis, the inventors performed LC-MS metabolomics on subcutaneous primary tumors and visceral metastatic nodules from the same NSG mice. These tumors arose from efficiently metastasizing melanomas derived from 4 different patients (M481, M405, M514, UT10). For each patient-derived xenograft, the inventors analyzed tumors from 3 different mice. In two independent experiments, unsupervised clustering of metabolomics data showed that metastatic nodules obtained from the liver, pancreas, and kidney always clustered together, distinct from subcutaneous tumors, irrespective of xenograft line (FIG. 7). Among the subcutaneous tumors and among metastatic nodules, samples clustered by patient. These data suggested there were consistent metabolic differences between subcutaneous primary tumors and visceral metastases as well as between xenografts derived from different patients.

One consistent difference between subcutaneous primary tumors and metastatic nodules was in the relative abundance of oxidized glutathione (GSSG) compared to reduced glutathione (GSH). GSH is one of the most abundant non-enzymatic cellular antioxidants, acting as a buffer to maintain redox homeostasis and prevent oxidative stress (Gorrini et al., 2013). The ratio of GSH to GSSG in metastatic nodules was significantly lower than in subcutaneous tumors (FIG. 3A). The GSH/GSSG ratio in circulating melanoma cells obtained from the blood was also significantly lower than in subcutaneous tumors from NSG mice (FIG. 3B). The lower GSH/GSSG ratio in circulating melanoma cells and metastatic nodules suggested that metastasizing cells experienced oxidative stress not observed in subcutaneous tumors, and that they consumed GSH in an effort to maintain redox homeostasis.

To test this, the inventors compared cytoplasmic and mitochondrial ROS levels in live melanoma cells from subcutaneous tumors, blood, and metastatic nodules in NSG mice transplanted with melanomas derived from patients M405, M481, and UT10. Cytoplasmic ROS levels were always significantly higher in circulating melanoma cells and visceral metastatic nodules as compared to subcutaneous tumors (FIG. 3C). Mitochondrial ROS levels were always significantly higher in visceral metastatic nodules as compared to circulating melanoma cells and subcutaneous tumors (FIG. 3D). ROS levels thus increased sharply in both circulating melanoma cells and metastatic nodules as compared to primary subcutaneous tumors, consistent with the decline in GSH/GSSG ratios in these cells.

One of the main sources of ROS is mitochondrial respiration. Mitochondrial mass declined significantly in circulating melanoma cells and metastatic nodules as compared to subcutaneous tumors (FIG. 3E). Mitochondrial membrane potential also declined significantly in circulating melanoma cells as compared to subcutaneous tumors but not in metastatic nodules (FIG. 3F). These data raised the possibility that melanoma cells reduced mitochondrial mass and mitochondrial function in circulating melanoma cells in an effort to reduce ROS generation.

To assess whether this reflected a reversible change, the inventors assessed mitochondrial mass in melanoma cells from the experiments that tested the reversibility of tumor-forming efficiency in different sites (shown in FIGS. 2C-E). Subcutaneous tumors always exhibited significantly higher mitochondrial mass as compared to metastatic nodules, irrespective of whether the subcutaneous tumors arose from the transplantation of subcutaneous tumor cells, circulating cells, or metastatic cells (FIG. 3G). Human melanoma cells thus undergo reversible changes in mitochondrial mass that are associated with changes in oxidative stress during metastasis.

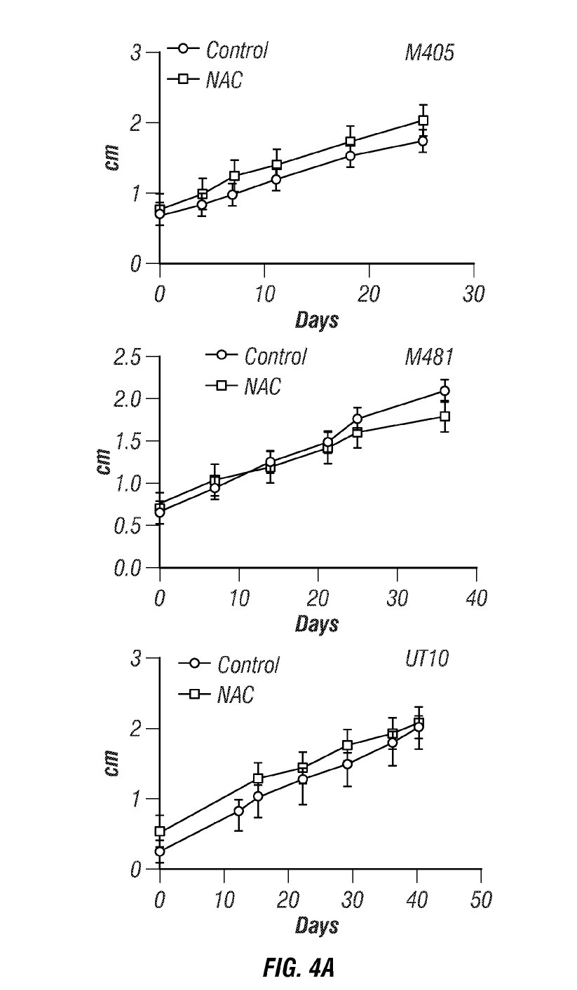

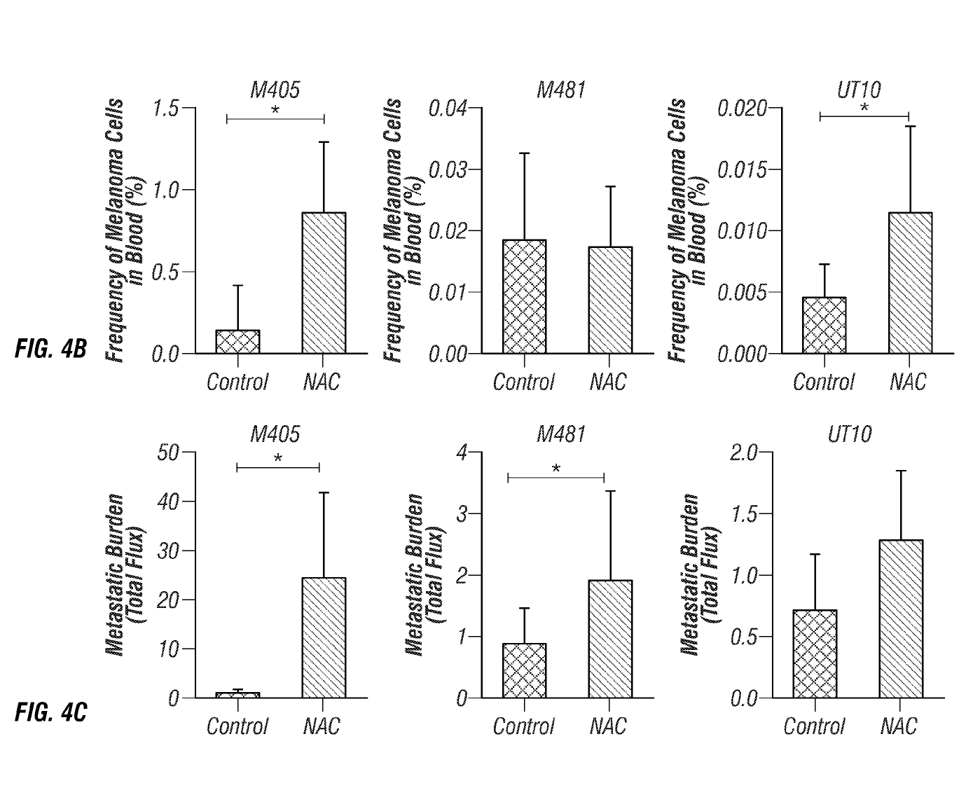

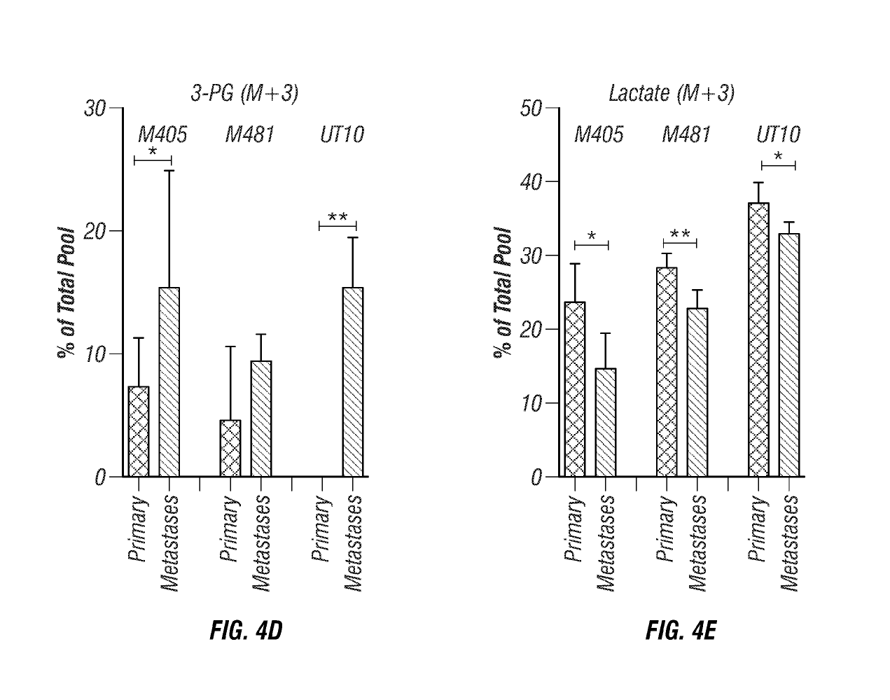

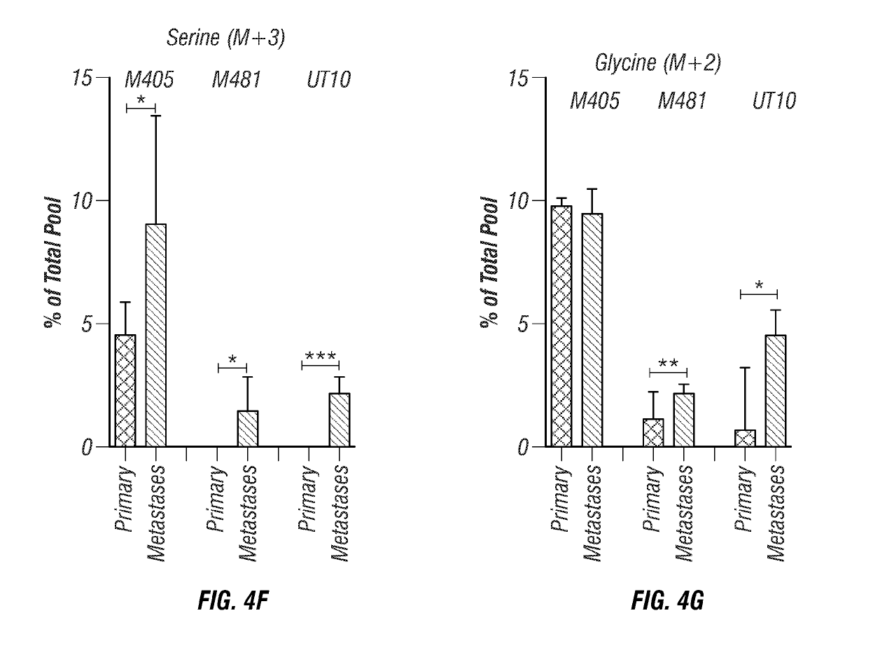

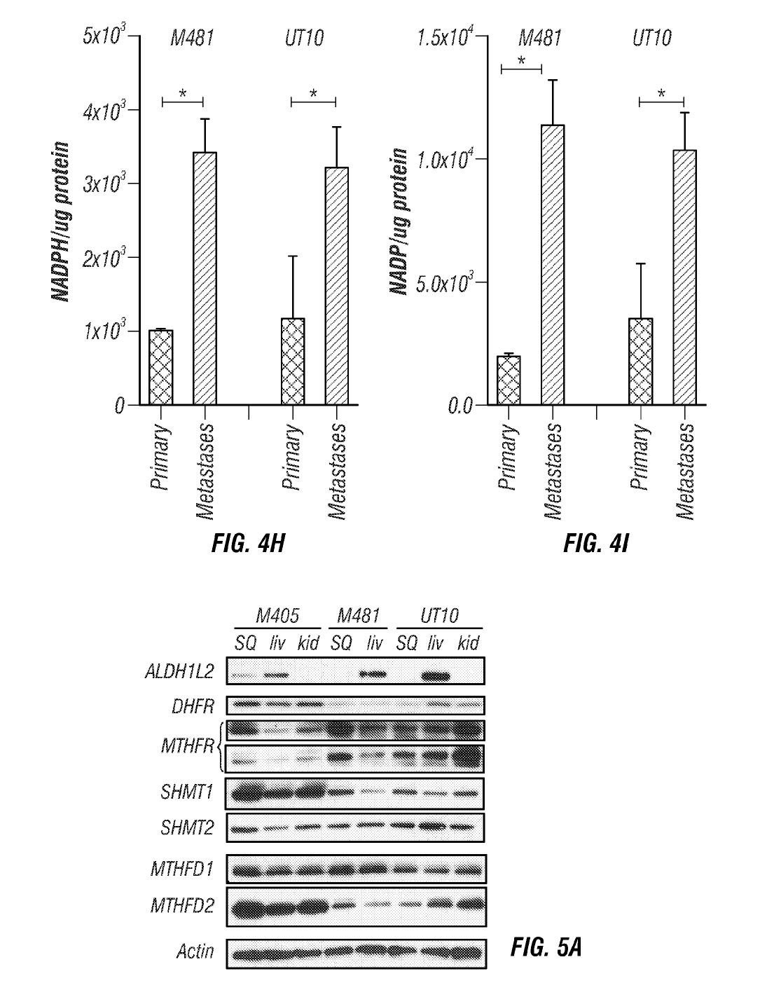

To test whether oxidative stress limits melanoma metastasis, the inventors transplanted efficiently metastasizing melanoma cells derived from three different patients (M405, M481, and UT10) into NSG mice and treated the mice with daily subcutaneous injections of the anti-oxidant N-acetyl-cysteine (NAC) (200 mg/kg/day). In no case did NAC treatment significantly affect the growth of primary subcutaneous tumors (FIG. 4A) but it significantly increased the frequency of melanoma cells in the blood of mice transplanted with M405 and UT10 (FIG. 4B) and significantly increased metastatic disease burden in mice transplanted with M405 and M481 (FIG. 4C). Oxidative stress therefore limits the metastasis of melanoma cells in vivo.

Metabolic Adaptations During Metastasis.

The inventors hypothesized that successfully metastasizing cells undergo reversible metabolic changes that allow them to withstand oxidative stress. One adaptation that could promote survival would be increased regeneration of GSH to increase the buffering capacity against oxidative stress (Gorrini et al., 2013). NADPH is needed to convert GSSG into GSH and increased production of NADPH promotes the regeneration of GSH (Gorrini et al., 2013). They assessed NADPH and NADP levels in melanoma cells from subcutaneous tumors and metastatic nodules obtained from NSG mice transplanted with melanomas derived from patients M481 and UT10. In both cases, the inventors observed significantly higher levels of NADPH and NADP in the metastatic cells as compared to primary subcutaneous tumor cells (FIGS. 4H-I). This raised the possibility that metastasizing cells generate higher levels of NADPH to increase their capacity to regenerate GSH in the face of oxidative stress.

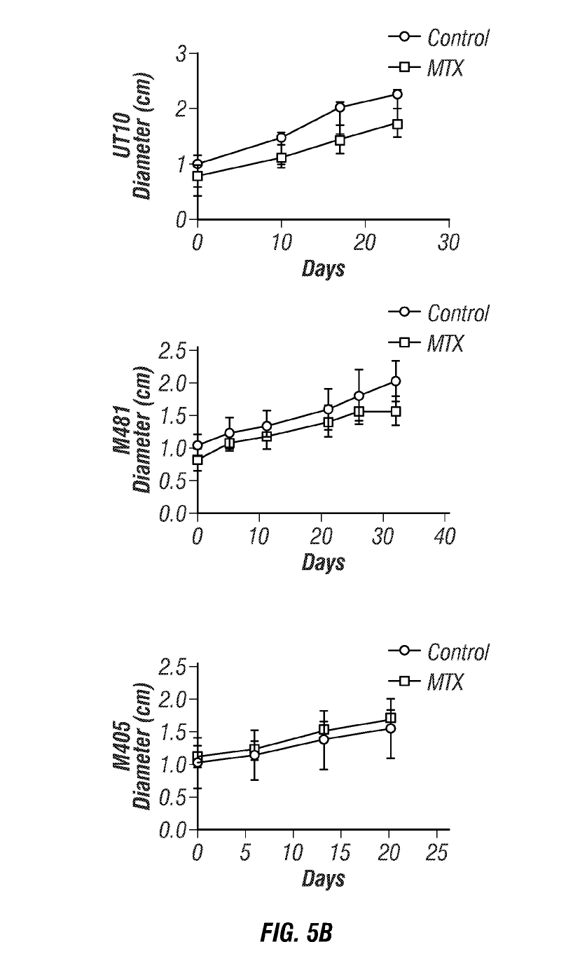

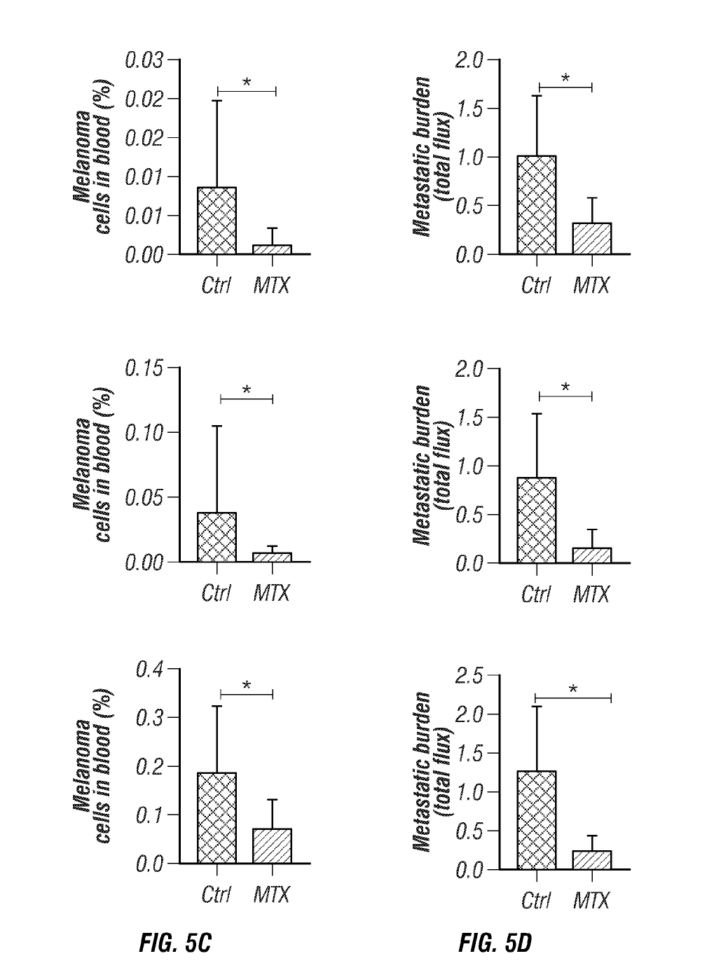

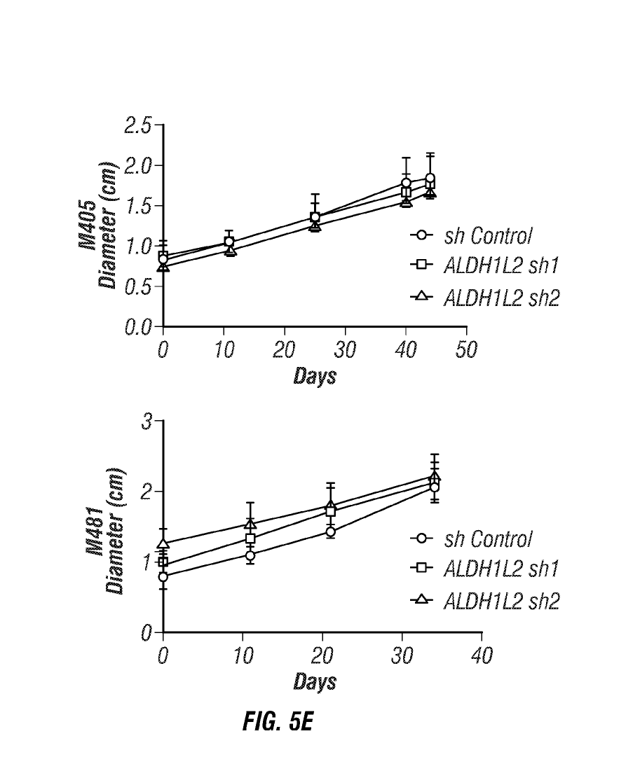

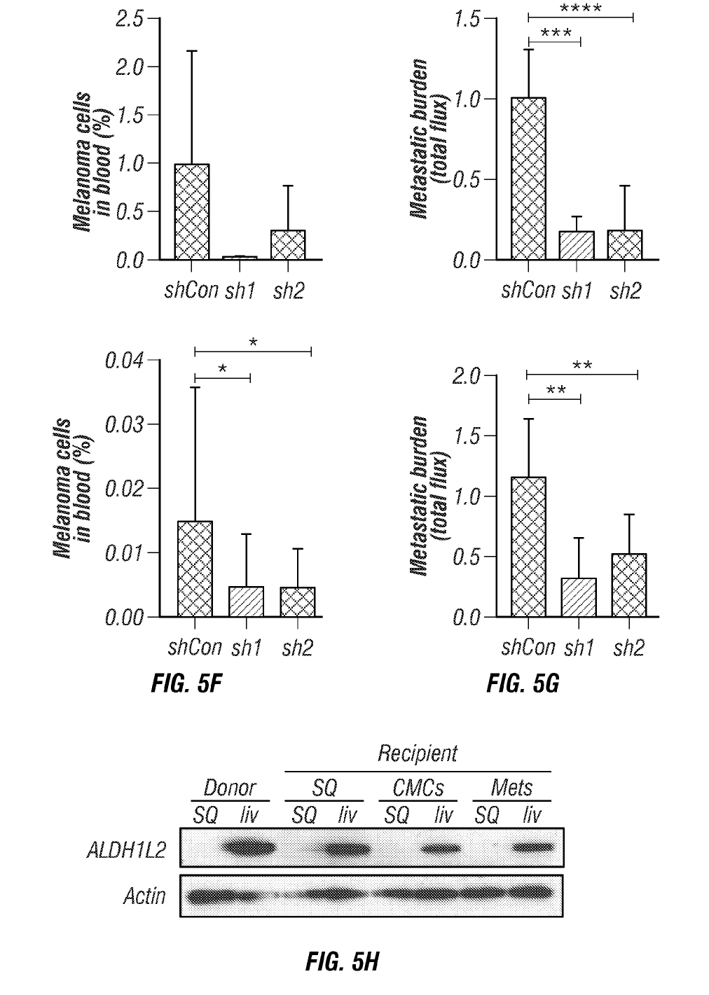

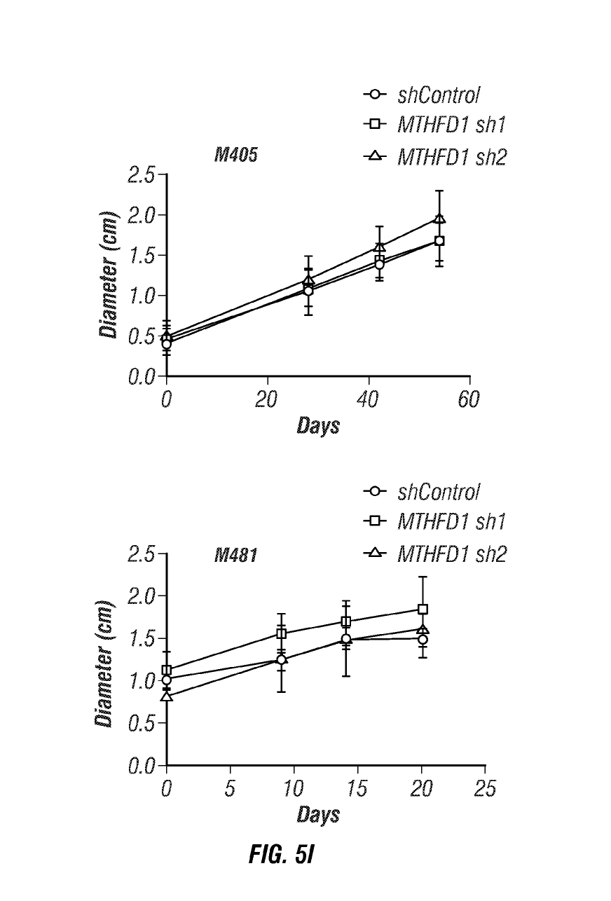

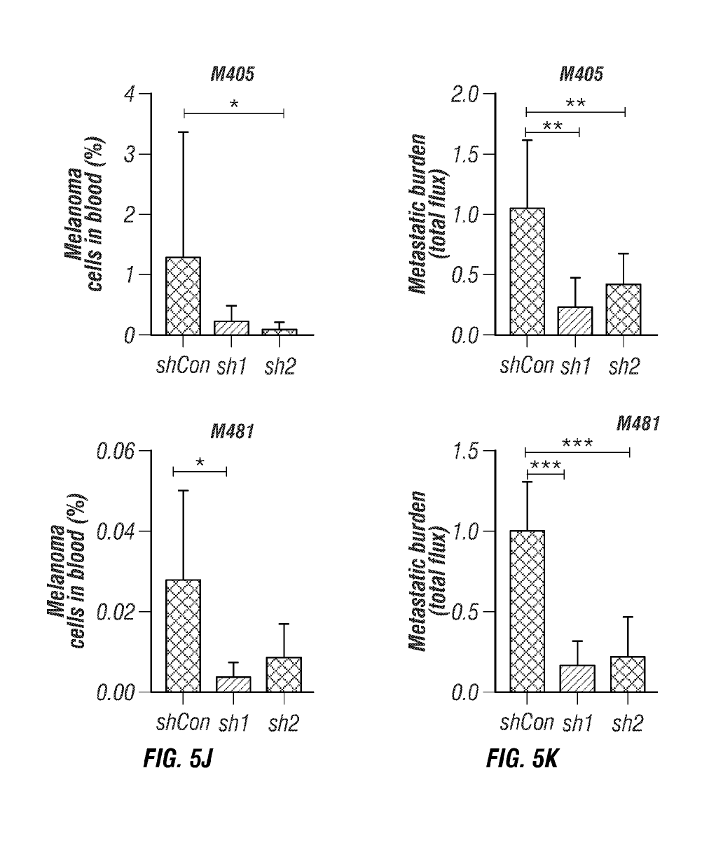

Folate metabolism was recently recognized as a major source of NADPH for oxidative stress management in cultured cell lines (Lewis et al., 2014, Fan et al., 2014 and Ye et al., 2014), raising the question of whether it can promote distant metastasis by primary cells in vivo. NADPH production from folate metabolism relies on conversion of serine to glycine and donation of a methyl group to tetrahydrofolate (FIG. 5A). Serine can be produced from glucose via the glycolytic intermediate 3-phosphoglycerate, and elevated de novo serine synthesis promotes the growth of some melanomas and breast cancers (Locasale et al., 2011 and Possemato et al., 2011). To test if carbon flux through this pathway changes during metastasis, the inventors administered ¹³C labeled glucose to NSG mice that had been transplanted with melanomas derived from patients M405, M481, and UT10. In metastatic tumors, the inventors, the inventors observed increased ¹³C labeling of 3-phosphoglycerate (FIG. 4D), but decreased ¹³C labeling of lactate (FIG. 4E), compared to subcutaneous tumors. They also observed increased ¹³C labeling of serine and glycine in metastatic tumors compared to subcutaneous tumors (FIGS. 4F-G). Metastatic tumors therefore demonstrate enhanced contribution of glucose carbon to de novo serine synthesis and the folate pathway relative to primary subcutaneous tumors.

Western blotting of key enzymes in the folate pathway showed a striking increase in the expression of the NADPH-producing enzymes ALDH1L1 and ALDH1L2 in liver metastases compared to subcutaneous tumors (FIG. 5A). Some metastases also showed reduced expression of an NADPH-consuming folate metabolism enzyme, MTHFR (FIG. 5A). The expression of other folate metabolism enzymes did not change consistently during metastasis. Some melanoma cells thus increase their expression of certain folate pathway enzymes that generate NADPH during metastasis.

To assess whether the increased ALDH1L1 and ALDH1L2 expression in metastases is reversible, the inventors compared the levels of ALDH1L1 and ALDH1L2 in primary subcutaneous tumors versus metastatic nodules from recipient mice transplanted with melanoma cells from subcutaneous tumors, the blood, or metastatic nodules in donor mice. ALDH1L1 and ALDH1L2 were much more highly expressed in liver nodules than primary subcutaneous tumors in both donor and recipient mice (FIG. 5H). This was true irrespective of whether the recipient tumors derived from melanoma cells from subcutaneous tumors, the blood, or metastatic nodules. This indicates that these changes in ALDH1L1 and ALDH1L2 expression in liver metastases as compared to subcutaneous tumors are reversible (FIG. 5H).

To test whether blocking the folate pathway inhibits metastasis, the inventors transplanted M405, M481 and UT10 melanoma cells subcutaneously in NSG mice and treated with a low dose of methotrexate (1.25 mg/kg/day), an inhibitor of dihydrofolate reductase, with simultaneous provision of thymidine (3 mg/kg/day) and hypoxanthine (750 μg/mg/day), to ameliorate the effects of folate pathway inhibition on nucleotide metabolism. Methotrexate treatment in these conditions had no significant effect on the growth of primary subcutaneous tumors, indicating that general cell proliferation was not impaired (FIG. 5B); however, the frequency of circulating melanoma cells in the blood of the same mice was significantly reduced (FIG. 5C) as well as metastatic disease burden (FIG. 5D). Metastasizing melanoma cells are therefore particularly sensitive to inhibition of folate metabolism, particularly those branches that produce NADPH, consistent with these data that melanoma cells upregulate this pathway during metastasis.

Folate metabolism is a major carbon contributor to nucleotide biosynthesis and to methylation reactions via production of methionine. However, the reactions catalysed by the cytoplasmic ALDH1L1 and the mitochondrial ALDH1L2 enzymes produce NADPH without contributing to nucleotide metabolism or methionine generation, and depletion of ALDH1L2 can decrease the GSH/GSSG ratio in vitro (Fan et al., 2014). To test whether ALDH1L2 is required for metastasis, the inventors identified two shRNAs that knocked down ALDH1L2 expression in melanoma cells derived from patient M405. They infected M405 and M481 melanoma cells with either shRNA or with a scrambled control shRNA then injected the cells subcutaneously in NSG mice. Neither shRNA against ALDH1L2 significantly affected the growth of primary subcutaneous tumors (FIG. 5E) but each significantly reduced circulating melanoma cell frequency and metastatic disease burden (FIGS. 5F and G). ALDH1L2 thus promotes metastasis in some melanomas in vivo. The inventors also tested the effect of MTHFD1 depletion on melanoma metastasis. They infected M405 and M481 with two shRNAs or with a scrambled shRNA and injected cells subcutaneously into NSG mice. Neither shRNA has a significant effect on subcutaneous tumor growth (FIG. 5I) but both shRNAs significantly reduced circulating melanoma cell frequency (FIG. 5J) and metastatic burden (FIG. 5K) in both melanomas.

### Example 3—Discussion

The mutations that are enriched among metastatic as compared to primary tumors tend to activate the same oncogenic pathways that promote primary tumor growths. Nonetheless, a subset of metastasis-associated gene products in breast cancer preferentially promote growth in metastatic sites (Minn et al., 2005, Bos et al., 2009, Kang et al., 2003, Oskarsson et al., 2011 and Padua et al., 2008). In melanoma, a number of gene products or mutations are significantly altered in metastatic as compared to primary tumors and regulate migration or invasion in culture (Scott et al., 2011, Kabbarah et al., 2010 and Kim et al., 2006). Most of these are oncogenes that would also be expected to promote proliferation and primary tumor growth. For example, Wnt pathway activation, such as by ß-catenin stabilization, promotes the development and growth of cutaneous melanomas as well as their metastasis (Delmas et al., 2007 and Grossmann et al., 2013). Metastasis is also regulated non-cell-autonomously by interactions with stromal cells (Oskarsson et al., 2011, Padua et al., 2008 and Oskarsson et al., 2014) and immune cells (Hanahan & Coussens, 2012).

The inability to identify metastasis-specific recurrent driver mutations (Vanharanta & Massague 2013 and Jones et al., 2008) calls into question the extent to which metastasis is driven by genetic as compared to epigenetic or metabolic changes. Epigenetic mechanisms or changes have been observed to regulate metastasis (Gupta et al., 2010, Fang et al., 2011, Ross-Innes et al., 2012 and Vanharanta et al., 2013). Metabolomic differences have been observed between primary and metastatic prostate tumors (Sreekumar et al., 2009) and breast cancer cells undergo metabolic changes during invasion or metastasis (Dong et al., 2013, Dong et al., 2013, Qu et al., 2011, Chen et al., 2007 and Lu et al., 2010) though it remains unclear to what extent these changes influence distant metastasis in vivo. This study suggests that metastasizing cancer cells must undergo metabolic changes to cope with oxidative stress. These results suggest that the reason why few circulating cancer cells are able to survive and proliferate (Luzzi et al., 1998, Cameron et al., 2000 and Kienast et al., 2010), and why distant metastasis is such an inefficient process (Vanharanta & Massague 2013), is that oxidative stress kills most metastasizing cancer cells. The results further suggest that metabolic pathways that generate NAPDH and buffer oxidative stress represent therapeutic targets to impede distant metastasis.

ROS can cause oncogenic mutations and activate oncogenic pathways, raising the possibility that treatment with anti-oxidants could suppress the initiation or progression of cancer (Gorrini et al., 2013 and Chandel & Tuveson, 2014). Antioxidants do appear to suppress the initiation of cancer in some contexts (Gao et al., 2007, Teoh-Fitzgerald et al., 2014 and Glasauer & Chandel, 2014). However, increasing dietary antioxidants has generally not reduced cancer incidence in clinical trials (Fortmann et al., 2013). In fact, consistent with these experiments, several studies have suggested that antioxidants can promote the initiation or progression of cancer. Deletion of NRF2 increases ROS and oxidative stress, impairing the formation and growth of tumors in mouse models of lung and pancreatic cancer (DeNicola et al., 2011). Inhibition of SOD1 also increases ROS levels and reduces tumor burden in a mouse model of lung cancer (Glasauer et al., 2014). The antioxidants NAC and Vitamin E accelerate cancer progression in mouse models of lung cancer (Sayin et al., 2014). Multiple clinical trials have found that dietary supplementation with anti-oxidants actually increases incidence and death from lung and prostate cancer (The Alpha-Tocopherol, Beta Carotene Cancer Prevention Study Group. The New England Journal of Medicine 330:1029-1035, 1994, Klein et al., 2011 and Goodman et al., 2004). In some cases the effects were sufficiently dramatic that trials were stopped early. Dietary supplementation with folate promotes the development and progression of breast cancer in rats and in patients (Deghan Manshadi et al., 2014 and Ebbing et al., 2009). These studies have been interpreted to suggest that anti-oxidants can promote cancer by reducing the load of deleterious mutations, by stimulating certain oncogenic pathways, or by inactivating certain tumor suppressors, though the precise mechanisms have remained obscure (Chandel & Tuveson, 2014). These results suggest that anti-oxidants promote metastasis and disease progression, at least in melanoma, by promoting the survival of metastasizing cells, increasing the efficiency of distant metastasis.

Although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### V. REFERENCES

The following references, to the extent that they provide exemplary procedural or other details supplementary to those set forth herein, are specifically incorporated herein by reference.
Bos et al., Nature, 459:1005-1009, 2009.
Cameron et al., Cancer Research, 60:2541-2546, 2000.
Chandel & Tuveson, The New England Journal of Medicine, 371:177-178, 2014.
Chen et al., Cancer Research, 67:1472-1486, 2007.
Debnath & Brugge, Nature Reviews Cancer, 5:675-688, 2005.
Debnath et al., Cell, 111:29-40, 2002.
Deghan Manshadi et al., PloS one, 9:e84635, 2014.
Delmas et al., Genes & Development, 21:2923-2935, 2007.
DeNicola et al., Nature, 475:106-109, 2011.
Dong et al., Cancer Cell, 23:316-331, 2013.
Ebbing et al., JAMA, 302:2119-2126, 2009.
Engell, Annals of Surgery, 149:457-461, 1959.
Fan et al., Nature, 510:298-302, 2014.
Fang et al., Science Translational Medicine, 3:75ra25, 2011.
Fortmann et al., In Vitamin, Mineral, and Multivitamin Supplements for the Primary
Prevention of Cardiovascular Disease and Cancer: A Systematic Evidence Review for the U.S. Preventive Services Task Force U.S. Preventive Services Task Force Evidence Syntheses, 2013.
Gao et al., Cancer Cell, 12:230-238, 2007.
Glasauer & Chandel, Biochemical Pharmacology, 92:90-101, 2014.
Glasauer et al., The Journal of Clinical Investigation 124, 117-128, (2014).
Goodman et al., Journal of the National Cancer Institute, 96:1743-1750, 2004.
Gorrini et al., Nature reviews, Drug Discovery, 12:931-947, 2013.
Griffiths et al., Cancer, 31:226-236, 1973.
Grossmann et al., Science Signaling, 6:ra14, 2013.
Gupta et al., Nature, 464:1071-1076, 2010.
Hanahan & Coussens, Cancer Cell, 21:309-322, 2012.
Hu & Smyth, Journal of Immunological Methods, 347:70-78, 2009.
Ishikawa et al., Science, 320:661-664, 2008.)
Jeffs et al., PloS one, 4:e8461, 2009.
Jones et al., Proceedings of the National Academy of Sciences USA, 105:4283-4288, 2008.
Joosse et al., EMBO Molecular Medicine, 2014.
Kabbarah et al., PloS one, 5:e10770, 2010.
Kamarajugadda et al., Cell Death & Disease, 4:e504, 2013.
Kang et al., Cancer Cell, 3:537-549, 2003.
Kienast et al., Nature Medicine, 16:116-122, 2010.
Kim et al., Cell, 125:1269-1281, 2006.
Klein et al., JAMA, 306:1549-1556, 2011.
LeBleu et al., Nature Cell Biology, 16:992-1003, 1001-1015, 2014.
Lewis et al., Molecular Cell, 55:253-263, 2014.
Locasale et al., Nature Genetics, 43:869-874, 2011.
Lu et al., The Journal of Biological Chemistry, 285:9317-9321, 2010.
Luzzi et al., The American Journal of Pathology, 153:865-873, 1998.
Minn et al., Nature, 436:518-524, 2005.
Mullen et al., Cell Reports, 7:1679-1690, 2014.
Nagrath et al., Nature, 450:1235-1239, 2007.
Oskarsson et al., Cell, 14:306-321, 2014.
Oskarsson et al., Nature Medicine, 17:867-874, 2011.
Padua et al., Cell, 133:66-77, 2008.
Porporato et al., Cell Reports, 8:754-766, 2014.
Possemato et al., Nature, 476:346-350, 2011.
Qu et al., The Journal of Clinical Investigation, 121:212-225, 2011.
Quintana et al., Science Translational Medicine, 4:159ra149, 2012.
Ross-Innes et al., Nature, 481:389-393, 2012.
Salsbury, Cancer Treatment Reviews, 2:55-72, 1975.
Sayin et al., Science Translational Medicine, 6:221ra215, 2014.
Schafer et al., Nature, 461:109-113, 2009.
Scott et al., Cancer Cell, 20:92-103, 2011.
Sreekumar et al., Nature, 457:910-914, 2009.
Stott et al., Science Translational Medicine, 2:25ra23, 2010.
Teoh-Fitzgerald et al., Oncogene, 33:358-368, 2014.)
The Alpha-Tocopherol, Beta Carotene Cancer Prevention Study Group. The New England Journal of Medicine 330:1029-1035, 1994.
Vanharanta & Massague, Cancer Cell, 24:410-421, 2013.
Vanharanta et al., Nature Medicine, 19:50-56, 2013.
Xia et al., Nucleic Acids Research, 40:W127-133, 2012.
Ye et al., Cancer Discovery, 4:1406-1417, 2014.
Yu et al., Science, 345:216-220, 2014.
Yu et al., Science, 339:580-584, 2013.

1. A method of inhibiting early metastasis in a subject having melanoma or breast cancer comprising administering to said subject an inhibitor of ALDH1L1, ALDH1L2, or MTHFD1.

2. The method of claim 1, wherein said subject is a human.

3. The method of claim 1, wherein said subject is a non-human mammal.

4. The method of claim 1, wherein inhibiting comprises reducing the number metastatic lesions, inhibiting the growth of metastases, killing metastatic melanoma cells in lesions or in circulation, inducing remission, extending remission, inhibiting recurrence or inhibiting progression.

5. The method of claim 1, wherein said melanoma is AJCC late state II or stage III disease.

6. The method of claim 1, wherein said melanoma is AJCC stage IV disease.

7. The method of claim 1, wherein said subject has previously received a radiotherapy, a chemotherapy, an immunotherapy, a molecularly targeted therapy or had surgical resection of a tumor.

8. The method of claim 1, wherein said inhibitor is an interfering RNA.

9. The method of claim 1, further comprising administering a second anti-melanoma therapy.

10. The method of claim 9, wherein the second anti-melanoma therapy is a MEK inhibitor, a BRAF and/or a cardiac glycoside is administered before the other agents.

11-14. (canceled)

15. The method of claim 1, wherein administering comprises topical, intravenous, intraarterial, subcutaneous, oral or intra-tumoral administration.

16. The method of claim 1, wherein administering comprises local, regional or systemic administration.

17. The method of claim 1, wherein administering comprises continuous infusion over a period of time.

18. The method of claim 1, wherein said subject has failed one or more standard melanoma therapies.

19-20. (canceled)

21. The method of claim 1, further comprising treating said subject with an immunomodulator.

22-24. (canceled)

25. The method of claim 9, wherein said second anti-melanoma therapy is a chemotherapeutic agent.

26. (canceled)

27. The method of claim 1, wherein said melanoma does not exhibit a pathogenic BRAF mutation.

28. The method of claim 1, wherein said melanoma exhibits a pathogenic BRAF mutation.

29. (canceled)

30. The method of claim 9, wherein said second melanoma therapy is a folate inhibitor.

31. A method of inhibiting melanoma early metastasis, inhibiting melanoma micrometastais, inhibiting melanoma progression and/or killing circulating melanoma cells in a subject having melanoma or breast cancer comprising administering to said subject an anti-folate.

32-60. (canceled)


SEQUENCE LISTING (4 sequences)

SEQ ID NO: 1 - DNA, 37 bp, Artificial sequence
  Synthetic primer
cgactctagaggatccatggatagcactgagaacgtc

SEQ ID NO: 2 - DNA, 67 bp, Artificial sequence
  Synthetic primer
tccacgtctccagcctgcttcagcaggctgaagttagtagctccgcttccctggaacaggtggtggc

SEQ ID NO: 3 - DNA, 74 bp, Artificial sequence
  Synthetic primer
gcctgctgaagcaggctggagacgtggaggagaaccctggacctggatccatggaagacgccaaaaacataaag

SEQ ID NO: 4 - DNA, 36 bp, Artificial sequence
  Synthetic primer
gcttgatatcgaattcttacacggcgatctttccgc